(19) United States
(12) Patent Application Publication
(10) Pub. No.: US 2006/0051493 A1
(43) Pub. Date: Mar. 9, 2006

(54) Apparatus and methods for printing arrays

(76) Inventors: Richard P. Tella, Sunnyvale, CA (US); William D. Fisher, San Jose, CA (US); Michael P. Caren, Palo Alto, CA (US); Lawrence J. DaQuino, Los Gatos, CA (US); Peter G. Webb, Menlo Park, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/630,490

(22) Filed: Jul. 29, 2003

Related U.S. Application Data
(60) Provisional application No. 60/400,347, filed on Jul. 31, 2002.

(51) Int. Cl. A61L 33/00; B05D 3/00; B05C 11/00

(52) U.S. Cl. 427/2.1; 427/8; 118/697; 118/713

(57) ABSTRACT

Apparatus and methods are disclosed for printing a plurality of biopolymer features on the surfaces of substrates. An apparatus comprises a substrate mount for receiving a substrate, a dispensing device for dispensing reagents for synthesizing a biopolymer on a surface of the substrate, an optical system for positioning the substrate mount along a y-axis and an optical system for positioning the dispensing device along an x-axis. The apparatus may comprise a touch system for positioning the substrate and the dispensing device along a z-axis. One of the substrate mount or the dispensing device is adapted for translation along the y-axis and for rotation about a central axis that is parallel to a z-axis. The other of the above is adapted to move along the x-axis transversely to the direction of, and independently of, movement of the one adapted for translation along the y-axis. The optical systems cooperate to position the substrate mount and the dispensing device relative to one another. Also disclosed in conjunction with the apparatus and methods are washing stations and loading stations.

### CROSS-REFERENCE TO RELATED APPLICATIONS

This application claims the benefit of the subject matter disclosed in prior copending Provisional Patent Application Ser. No. 60/600,437 filed Jul. 31, 2003, the disclosure of which is incorporated herein by reference in its entirety.

### BACKGROUND OF THE INVENTION

This invention relates to the manufacture of substrates or supports having bound to the surfaces thereof a plurality of chemical compounds, such as biopolymers. In one aspect the invention relates to the manufacture of arrays formed and arranged by depositing compounds or synthesizing large numbers of compounds on solid substrates in a predetermined arrangement. In another aspect this invention relates to the field of bioscience in which arrays of oligonucleotide probes are fabricated or deposited on a surface and are used to identify or analyze DNA sequences in cell matter. The present invention has a wide range of application for synthesis and use of arrays of oligonucleotides or proteins for conducting cell study, for diagnosing disease, identifying gene expression, monitoring drug response, determination of viral load, identifying genetic polymorphisms, and the like.

In the field of diagnostics and therapeutics, it is often useful to attach species to a surface. One important application is in solid phase chemical synthesis wherein initial derivatization of a substrate surface enables synthesis of polymers such as oligonucleotides and peptides on the substrate itself. Substrate bound oligomer arrays, particularly oligonucleotide arrays, may be used in screening studies for determination of binding affinity. Modification of surfaces for use in chemical synthesis has been described. See, for example, U.S. Pat. No. 5,624,711 (Sundberg), U.S. Pat. No. 5,266,222 (Willis) and U.S. Pat. No. 5,137,765 (Farnsworth).

Determining the nucleotide sequences and expression levels of nucleic acids (DNA and RNA) is critical to understanding the function and control of genes and their relationship, for example, to disease discovery and disease management. Analysis of genetic information plays a crucial role in biological experimentation. This has become especially true with regard to studies directed at understanding the fundamental genetic and environmental factors associated with disease and the effects of potential therapeutic agents on the cell. Such a determination permits the early detection of infectious organisms such as bacteria, viruses, etc.; genetic diseases such as sickle cell anemia; and various cancers. This paradigm shift has lead to an increasing need within the life science industries for more sensitive, more accurate and higher-throughput technologies for performing analysis on genetic material obtained from a variety of biological sources.

Unique or misexpressed nucleotide sequences in a polynucleotide can be detected by hybridization with a nucleotide multimer, or oligonucleotide, probe. Hybridization is based on complementary base pairing. When complementary single stranded nucleic acids are incubated together, the complementary base sequences pair to form double stranded hybrid molecules. These techniques rely upon the inherent ability of nucleic acids to form duplexes via hydrogen bonding according to Watson-Crick base-pairing rules. The ability of single stranded deoxyribonucleic acid (ssDNA) or ribonucleic acid (RNA) to form a hydrogen bonded structure with a complementary nucleic acid sequence has been employed as an analytical tool in molecular biology research. An oligonucleotide probe employed in the detection is selected with a nucleotide sequence complementary, usually exactly complementary, to the nucleotide sequence in the target nucleic acid. Following hybridization of the probe with the target nucleic acid, any oligonucleotide probe/nucleic acid hybrids that have formed are typically separated from unhybridized probe. The amount of oligonucleotide probe in either of the two separated media is then tested to provide a qualitative or quantitative measurement of the amount of target nucleic acid originally present.

Direct detection of labeled target nucleic acid hybridized to surface-bound polynucleotide probes is particularly advantageous if the surface contains a mosaic of different probes that are individually localized to discrete, and often known, areas of the surface. Such ordered arrays containing a large number of oligonucleotide probes have been developed as tools for high throughput analyses of genotype and gene expression. Oligonucleotides synthesized on a solid substrate recognize uniquely complementary nucleic acids by hybridization, and arrays can be designed to define specific target sequences, analyze gene expression patterns or identify specific allelic variations. The arrays may be used for conducting cell study, diagnosing disease, identifying gene expression, monitoring drug response, determination of viral load, identifying genetic polymorphisms, analyzing gene expression patterns or identifying specific allelic variations, and the like.

In one approach, cell matter is lysed, to release its DNA as fragments, which are then separated out by electrophoresis or other means, and then tagged with a fluorescent or other label. The resulting DNA mix is exposed to an array of oligonucleotide probes, whereupon selective binding to matching probe sites takes place. The array is then washed and interrogated to determine the extent of hybridization reactions. In one approach the array is imaged so as to reveal for analysis and interpretation the sites where binding has occurred. Arrays of different chemical compounds or moieties or probe species provide methods of highly parallel detection, and hence improved speed and efficiency, in assays. Assuming that the different sequence polynucleotides were correctly deposited in accordance with the predetermined configuration, then the observed binding is indicative of the presence and/or concentration of one or more polynucleotide components of the sample.

The arrays may be microarrays created on the surface of a substrate by in situ synthesis of biopolymers such as polynucleotides, polypeptides, polysaccharides, etc., and combinations thereof, or by deposition of molecules such as oligonucleotides, cDNA and so forth. In general, arrays are synthesized on a surface of a substrate or substrate by one of any number of synthetic techniques that are known in the art. In one approach, for example, the substrate may be one on which a single array of chemical compounds is synthesized. Alternatively, multiple arrays of chemical compounds may be synthesized on the substrate, which is then diced, i.e., cut, into individual assay devices, which are substrates that each comprise a single array, or in some instances multiple arrays, on a surface of the substrate.

One of the steps in the synthesis process usually involves depositing small volumes of liquid containing reagents for the synthesis, for example, monomeric subunits or whole polynucleotides, onto to surface of a support or substrate. In one approach, a pin spotter is employed. A pin spotter uses one or more needles to transfer small volumes of fluid onto the surface of a substrate. There are several disadvantages with the use of pin spotters. First, they require that the pin be stopped in position before transferring liquid to the substrate, which makes the process inherently slow. Second, they need to be loaded frequently, which also tends to slow the process. Finally, their dispense volume varies with the amount of fluid loaded. In another approach, ink-jet printing has been proposed for use in depositing small volumes of liquid for synthesis of chemical compounds on the surface of substrates.

There remains a need, however, for an apparatus and process that would permit the use of ink-jet printing techniques with precision capability and with the capabilities of material handling and real time inspection to produce high quality arrays in volume at a low cost. The apparatus should allow accurate positioning and firing of print heads over the substrate to build the arrays. The apparatus should also provide for loading and maintenance of the print head, in-process inspection of the array printing, and all preliminary initialization and calibrations necessary to achieve the primary function.

### SUMMARY OF THE INVENTION

One embodiment of present invention is an apparatus comprising a substrate mount for receiving a substrate, a dispensing device for dispensing reagents for synthesizing a biopolymer on a surface of the substrate, an optical system for positioning the substrate mount along a y-axis and an optical system for positioning the dispensing device along an x-axis. Either the substrate mount or the dispensing device is adapted for translation along the y-axis and for rotation about a central axis that is parallel to a z-axis. The other of the above is adapted to move along the x-axis transversely to the direction of, and independently of, translation of whichever one of the substrate mount or dispensing device, which moves along the y-axis. The optical systems cooperate to position the substrate mount and the dispensing device relative to one another. Optionally, the apparatus may comprise a touch system for positioning the substrate and the dispensing device along a z-axis.

Another embodiment of the present invention is an apparatus for synthesizing a plurality of biopolymer features on the surface of a substrate. The apparatus comprises a substrate mount for receiving a substrate, a dispensing device for dispensing reagents for synthesizing biopolymers on a surface of the substrate, an optical system for positioning the substrate mount along a y-axis and an optical system for positioning the dispensing device along an x-axis, a touch system for positioning the substrate and the dispensing device along a z-axis, a loading station for loading the reagents into the dispensing device, a mechanism for moving the dispensing device and/or the loading station relative to one another, a wash station for washing the dispensing device, and a mechanism for moving the dispensing device and/or the wash station relative to one another. The substrate mount is adapted for translation along the y-axis and for rotation about a central axis that is parallel to a z-axis. The dispensing device moves along the x-axis transversely and independently with respect to the substrate mount. The optical systems cooperate to position the substrate mount and the dispensing device relative to one another.

Another embodiment of the present invention is a method comprising positioning a substrate along a y-axis by means of an optical system, positioning a dispensing device along an x-axis by means of an optical system, positioning the substrate and the dispensing device relative to one another along an orthogonal axis by means of at least one touch system, and depositing a reagent for synthesizing a biopolymer on a surface of the substrate by means of the dispensing device. The optical systems cooperate to independently position the substrate mount and the dispensing device relative to one another.

Another embodiment of the present invention is a method for synthesizing an array of biopolymers on a surface of a substrate. One or more polymer subunits are added, in multiple rounds of subunit additions, at each of multiple feature locations on the surface to form one or more arrays. In each round of subunit additions the substrate and a dispensing system for dispensing the polymer subunits for the synthesis of the biopolymers are independently brought into a dispensing position relative to the activated discrete sites on the surface. The substrate is positioned along a y-axis by means of an optical system and the dispensing system is positioned along an x-axis by means of an optical system. The substrate mount and the dispensing system are positioned relative to one another by means of the optical systems. The dispensing system is independently positioned along a z-axis by means of at least one touch system. The polymer subunits are dispensed to the discrete sites. The substrate and/or the dispensing system are removed from the relative dispensing position.

Another embodiment of the present invention is a loading apparatus for loading reagents into a dispensing device. The loading station comprises one or more reagent receptacles covered by a retractable cover. Usually, the loading apparatus comprises a retractable cover so that the cover remains on a housing of the loading apparatus until the loading apparatus is employed to add reagents to the dispensing device. The loading apparatus may be employed with any device for dispensing reagents to a surface such as a droplet dispensing device.

Another embodiment of the present invention is a wash apparatus for washing a reagent dispensing device. The wash apparatus comprises one or more wet wash pads for wet washing a surface or a portion of the reagent dispensing device and one or more dry pads for dry wiping the reagent dispensing device.

Another embodiment of the present invention is an apparatus comprising a substrate mount for receiving a substrate, a dispensing device for dispensing reagents for synthesizing a biopolymer on a surface of the substrate, and a touch system for positioning the substrate and the dispensing device along a z-axis.

### BRIEF DESCRIPTION OF THE DRAWINGS

FIG. 1 is a perspective view of a substrate bearing multiple arrays, as may be produced by a method and apparatus of the present invention.

FIG. 2 is an enlarged view of a portion of FIG. 1 showing some of the identifiable individual regions (or “features”) of a single array of FIG. 1.

FIG. 3 is an enlarged cross-section of a portion of FIG. 2.

FIG. 4 is a schematic representation of one embodiment of an apparatus in accordance with the present invention.

FIG. 5 is a perspective view of one portion of the apparatus of FIG. 4 depicting a substrate mount, print head and inspection system.

FIG. 6 is perspective view of the substrate of FIG. 1 taken from the bottom.

FIG. 7 is a perspective view of one portion of the apparatus of FIG. 4.

FIG. 8 is a perspective view of one portion of the apparatus of FIG. 4 depicting a loading station.

FIG. 9 is a depiction of a loading block of a loading station for use with an apparatus in accordance with the present invention.

FIG. 10 is a depiction in cross-section of a receptacle of a washing block of a wash station of the apparatus of FIG. 4.

FIG. 11A is a sketch of an embodiment of a loading block of a loading station of the apparatus of FIG. 8 in perspective view.

FIG. 11B is a sketch in a plan view of the embodiment of FIG. 11A.

FIG. 11C is a sketch in sectional view through 1-1′ of the embodiment of FIG. 11B, showing in wells from left to right the progress of forming a convex meniscus at the well opening by inward displacement of a portion of the wall, and showing at right the transfer of the liquid to a receptacle brought into contact with the convex meniscus.

FIG. 12 is a perspective view of one portion of the apparatus of FIG. 4 depicting a wash station.

FIG. 13 is a perspective view of one portion of the apparatus of FIG. 4 depicting a touch system.

### DETAILED DESCRIPTION OF THE INVENTION

In one aspect the present invention provides apparatus and methods for manufacturing substrates having a plurality of chemical compounds such as biopolymer features on a surface of the substrate. An apparatus generally comprises a substrate mount, an optical system associated with the substrate mount, a dispensing device, an optical system associated with the dispensing device, and a touch system. The substrate mount receives a substrate and the associated optical system assists in positioning the substrate along a y-axis and positioning the substrate and the dispensing device relative to one another along a y-axis. The dispensing device dispenses reagents for synthesizing a biopolymer on a surface of the substrate. The optical system associated with the dispensing device positions the dispensing device along an x-axis. A touch system positions the substrate and the dispensing device relative to one another along a z-axis. In a preferred embodiment the dispensing device moves transversely with respect to the substrate.

In the description herein the terms “x-axis,” “y-axis” and “z-axis” reference distinct axes and, preferably, a coordinate system that is orthogonal, i.e., a Cartesian coordinate system.

The phrase “optical system associated with” includes image sensors as well as circuitry, motors, processors, and the like, all of which cooperate to provide movement of some of the components of an apparatus of the invention usually independently of one another. An optical system associated with one element of the apparatus may utilize one or more features such as an image sensor, etc., of an optical system associated with another element of the apparatus when the latter element is not utilizing such features.

The phrase “adapted to” or “adapted for” is used herein with respect to components of the present apparatus. The components of the present apparatus are adapted to perform a specified function by a combination of hardware and software. This includes the structure of the particular component and may also, and usually does, include a microprocessor, embedded real-time software and I/O interface electronics to control the sequence of operations of the invention.

The components of the apparatus are normally mounted on a suitable frame in a manner consistent with the present invention. The frame of the apparatus is generally constructed from a suitable material that gives structural strength to the apparatus so that various moving parts may be employed in conjunction with the apparatus. Such materials for the frame include, for example, metal, lightweight composites, granite and the like.

The apparatus may also comprise a loading station for loading reagents into the dispensing device and a mechanism for moving the dispensing device and/or the loading station relative to one another. The apparatus may also comprise a wash station for washing the dispensing device and a mechanism for moving the dispensing device and/or the wash station relative to one another. The apparatus further may comprise a mechanism for inspecting the reagent deposited on the surface of the substrate.

The substrate mount may be any convenient structure on which the substrate may be placed and held for depositing reagents on the surface of the substrate. The substrate mount may be of any size and shape and generally has a shape similar to that of the substrate as long as it is sufficiently able to support the substrate. For example, the substrate mount is rectangular for a rectangular substrate, circular for a circular substrate and so forth. The substrate mount may be constructed from any material of sufficient strength to physically receive and hold the substrate during the deposition of reagents on the substrate surface as well as to withstand the rigors of movement in one or more directions. Such materials include metal, composites, and the like.

The support or substrate may be retained on the substrate mount by gravity, friction, vacuum, and the like. The surface of the substrate mount, on which the substrate is received, may be flat. On the other hand, and preferably, the surface of the substrate mount may comprise certain structural features such as, for example, parallel upstanding linear ribs, and the like, on which the substrate is placed. Whether the substrate mount is flat or comprises structural features, the resulting surface of the substrate mount on which the substrate rests is planar. The nature and number of structural features is generally determined by the size, weight and shape of the substrate, and so forth. In one embodiment the upper surface of the substrate mount has openings that communicate with a suitable vacuum source to hold the substrate on the substrate mount. The openings may be in the surface of the substrate mount or in structural features on the surface of the substrate mount. In a specific embodiment the substrate mount is a vacuum chuck.

The substrate mount is adapted for translation along a y-axis and also for rotation about a center axis that is parallel to a z-axis. Translation along a y-axis provides for moving a substrate on the substrate mount in position for dispensing of reagents to a surface of the substrate. Usually, this requires that the surface of the substrate be parallel to the surface of the dispensing device on which dispensing nozzles are located. Accordingly, the surface of the substrate is normal to the direction in which fluid is dispensed to the surface of the substrate. The ability of the substrate to rotate about a central axis allows the optical system associated with the substrate mount to provide accurate orientation of the substrate with respect to a dispensing device during the dispensing of reagents to the surface of the substrate.

In one approach the substrate mount is carried by a stage arrangement, which provides for the desired movement parameters independently of the movement of the dispensing device. In this approach the substrate mount is secured to the stage, which is usually attached to a frame member of the present apparatus. For example, the substrate mount may be carried by a stacked Increment-Theta stage arrangement that is attached directly to a granite base. Other approaches for providing the substrate mount with desired movement capabilities may be employed.

The fluid dispensing device normally includes a reagent source or manifold as well as reagent lines that connect the source to fluid dispensing nozzles and the like. Any system may be employed that dispenses fluids such as water, aqueous media, organic solvents and the like as droplets of liquid. The fluid dispensing device may comprises a pump for moving fluid and may also comprise a valve assembly and a manifold as well as a means for delivering predetermined quantities of fluid to the surface of a substrate. The fluids may be dispensed by any known technique. Any standard pumping technique for pumping fluids may be employed in the dispensing device. For example, pumping may be by means of a peristaltic pump, a pressurized fluid bed, a positive displacement pump, e.g., a syringe pump, and the like.

In one specific embodiment a droplet dispensing device comprises one or more heads, which may be of a type commonly used in an ink jet type of printer. Each head carries hundreds of ejectors or nozzles to deposit droplets. In the case of heads, each ejector may be in the form of an electrical resistor operating as a heating element under control of a processor (although piezoelectric elements could be used instead). Each orifice with its associated ejector and a reservoir chamber, acts as a corresponding pulse jet with the orifice acting as a nozzle. In this manner, application of a single electric pulse to an ejector causes a droplet to be dispensed from a corresponding orifice (or larger droplets could be deposited by using multiple pulses to deposit a series of smaller droplets at a given location). Certain elements of a suitable head can be adapted from parts of a commercially available thermal inkjet print head device available from Hewlett-Packard Co. as part no. HP51645A. However, other head configurations can be used as desired.

As is well known in the ink jet print art, the amount of fluid that is expelled in a single activation event of a pulse jet, can be controlled by changing one or more of a number of parameters, including the orifice diameter, the orifice length (thickness of the orifice member at the orifice), the size of the deposition chamber, and the size of the heating element, among others. The amount of fluid that is expelled during a single activation event is generally in the range about 0.1 to 1000 pL, usually about 0.5 to 500 pL and more usually about 1.0 to 250 pL. A typical velocity at which the fluid is expelled from the chamber is more than about 1 m/s, usually more than about 10 m/s, and may be as great as about 20 m/s or greater. As will be appreciated, if the orifice is in motion with respect to the receiving surface at the time an ejector is activated, the actual site of deposition of the material will not be the location that is at the moment of activation in a line-of-sight relation to the orifice, but will be a location that is predictable for the given distances and velocities.

The reagent dispensing device is adapted for translation along an x-axis independently of the movement of the substrate mount along the y-axis. Translation along an x-axis provides for moving the dispensing device transversely to the direction of movement of the substrate mount (along the y-axis) and in position for dispensing of reagents to the surface of a substrate. In one approach the reagent dispensing device is carried by a stage arrangement, which provides for the desired movement parameters. In this approach the dispensing device is secured to the stage, which is usually attached to a frame member of the present apparatus. For example, in one approach the dispensing device may be carried by an orthogonal z-axis stage arrangement attached to an x-axis stage arrangement, which is attached directly to a rigid supporting granite beam off a granite base to which the substrate mount is secured. Other approaches for providing the dispensing device with desired movement capabilities may be employed.

To achieve the desired level of dispensing accuracy, the substrate on the substrate mount should be oriented parallel to dispensing device on the y-axis. The positioning of the substrate mount relative to the dispensing device is accomplished using optical systems, which comprise at least one, and in some optical systems, more than one image sensor. Usually, an optical system is employed for positioning the substrate mount along the y-axis as described above. In this instance the optical system usually comprises at least two image sensors. An optical system is employed for positioning the dispensing device along the x-axis. In this instance the optical system usually comprises at least one image sensor. Thus, the optical systems are cooperative to position the dispensing device and the substrate mount relative to one another. Usually, the image sensor is part of a camera.

The physical dimensions of the cameras are determined by the overall space constraints of the present apparatus and the dimensions of the other components. Usually, two or more cameras are employed for positioning the substrate mount along the y-axis (including rotation of the substrate mount) where the number of cameras generally corresponds with the number of target images on the substrate that are viewed to achieve the desired predetermined orientation of the substrate with respect to the dispensing device. The number of target images is usually that which is sufficient to achieve accurate positioning of the substrate relative to the dispensing device. Usually, at least two target images are employed but three or more may be used. Likewise, the number of cameras employed for positioning the dispensing device along the x-axis generally corresponds to the number of target images (fiducials) on the dispensing device. The target images are placed on the dispensing device so that the respective cameras may view them. Usually, the target images are on the surface of the dispensing device on which the dispensing nozzles are located.

In one embodiment two cameras are associated with the substrate mount in such a manner as to view target images on the underside of the substrate, i.e., the side of the substrate that is opposite the side to which reagents are dispensed. Since the substrate lies on the substrate mount, the substrate mount comprises openings that correspond to the location of the cameras, which are mounted in such a manner as to view the target images through the openings. Generally, the cameras are mounted on a frame member on which the stage that carries the substrate mount is attached, usually, adjacent to the stage. The cameras are generally on opposing sides of the perimeter of the stage. The dimensions of the openings in the substrate mount are sufficient to permit viewing of the target images on the substrate. Usually, the openings are about the same size or slightly larger than the size of the target images and of the camera image sensor regions. Usually, the target images are placed equidistant from opposing edges of the substrate.

The target images may be any suitable image that can be sensed by the image sensors. The images may be of any convenient shape and dimension. The target images are placed on the substrate by any approach that produces an image that may be sensed by the image sensor. Such approaches include, for example, etching, deposition, and the like. The target images of the substrate are located on opposing sides of the substrate in such a manner that the image sensors of the respective cameras may view them. Since the target images are employed to maintain a predetermined orientation of the substrate on the substrate mount, they are placed on the substrate in precise locations to achieve the predetermined orientation. As mentioned above, the substrate mount is rotatable around a center axis (such as by employing a rotation stage as in the example above). Accordingly, when the cameras view the substrate through the openings in the substrate mount and the respective image sensors sense both target images at the correct position, the substrate on the substrate mount is in the desired orientation for accurate dispensing of reagents to the substrate surface. If the respective cameras do not sense the target images at the correct position, then the substrate mount is rotated automatically to bring the target images into the correct view by the respective cameras, thereby achieving the desired predetermined orientation of the substrate on the substrate mount. In one approach the cameras are in communication with a computer, which is in turn in communication with the substrate mount so that the rotation of the substrate mount to the desired position may be achieved as a result of the input from the computer to the mechanism for moving the substrate mount. The appropriate circuitry, motors, computers and the like to achieve the above are standard in the art and will not be discussed in detail herein.

An optical system associated with the dispensing device may include one or more image sensors for positioning the dispensing device along the x-axis. In one embodiment an image sensor is mounted on the frame on which the substrate mount is secured. The image sensor views one or more target images on the dispensing device placed for viewing by the image sensor. As above with the substrate, any suitable target image may be employed for sensing by the image sensor. The image sensor is in suitable communication with a computer, which communicates with a motor assembly to move the dispensing device.

As mentioned above, it is important to maintain the dispensing device a desired predetermined distance from the substrate on the substrate mount. One problem is that the substrates may have differences in thickness or other imperfections. Accordingly, if the dispensing device is set at a predetermined distance from the substrate on the substrate mount, the actual distance may change from one substrate to the next. In the present invention appropriate techniques are employed to provide accurate orientation of the fluid dispensing device even though variations in the substrate are present. A touch system is employed that usually comprises at least two opposing touch probes. One of the probes is an upwardly pointing probe and is affixed to a supporting member of the apparatus, usually, adjacent to the substrate mount and, more usually, on the same supporting member to which the substrate mount is affixed. The other of the touch probes is a downwardly pointing probe and is affixed to a frame member such as an arm that carries the dispensing device. The probes are generally constructed from any material suitable for such probes. The shape and dimensions of the probes are not critical. Usually, the probes are shaped in the form of a rod or the like. The length of the probes and thickness of the probes are such as to accomplish the desired function of the touch probes. The probes are linked by means of appropriate circuitry to a computer, which assists in adjusting the position and orientation of the dispensing device relative to the substrate mount to maintain a predetermined distance between the nozzles of the dispensing device and the surface of a substrate on the substrate mount. An image sensor is employed to observe the touch probes during calibration, set up and use. The image sensor may be part of a camera and communicates with a computer to allow proper positioning of the various devices.

In operation of the touch system, an initial calibration of the probes is made to set the dispensing device relative to the surface of the substrate at a predetermined height. Furthermore, the angle of the dispensing device is adjusted to assure a parallel relationship between the surface of the substrate and the nozzles on the dispensing device. In the present invention the upwardly pointing touch probe moves only along the y-axis, that is, the same axis along which the substrate mount moves. The downwardly pointing probe moves along the x-axis, that is, the same axis along which the dispensing device moves. As mentioned above, the dispensing device moves along the x-axis transversely to the direction of movement of the substrate mount and also moves vertically to permit adjustment of the distance between the nozzles of the dispensing device and the surface of the substrate mount. The upwardly depending probe touches a point on the bottom of the dispensing device assembly. The downwardly depending probe touches a point on the surface of the substrate mount or on the surface of the substrate. Adjustments are made to the position and angle of orientation of the dispensing device and of the nozzles of the dispensing device during this calibration and set up period. Usually, the touch probes are employed only during the calibration and set up procedure and adjustment for variations in thickness of the substrate is then made.

The present apparatus may also comprise a delivery device for delivering the substrate to the substrate mount. The delivery device has the function of receiving or removing a substrate from a substrate supply device and transporting the substrate to the substrate mount. Thus, the delivery device may have any convenient configuration, as long it is able to carry out the above functions. In one embodiment the delivery device is in the form of a two-prong fork where the supporting members (or prongs) of the fork are adapted to receive and carry the substrate. Usually, the prongs are designed to engage the underside surface of the substrate at the perimeter of the substrate. The delivery device may be made of any material that has the structural strength to carry the substrate and withstand the transport functions of the delivery device. Such materials include, for example, metals, lightweight composites, and so forth. The substrate may be retained on the substrate mount by gravity, friction, vacuum, and the like. In one embodiment the upper surface of the substrate mount has openings that communicate with a suitable vacuum source to hold the substrate on the substrate mount. The openings may be in the surface of the substrate mount or in structural features or support members on the surface of the substrate mount.

Another function of the delivery device is to deliver the substrate to the substrate mount so that preliminary adjustments may be made to provide the substrate to the substrate mount in a desired predetermined orientation. In this way the optical system of the substrate mount needs only to fine tune the orientation thereby achieving the desired predetermined orientation of the substrate relative to the dispensing device. To this end, the delivery device has associated therewith a delivery device optical system for positioning the substrate along an x-axis and a y-axis. The optical system may be similar in design to that discussed above for the substrate mount optical system. Thus, the delivery device optical system may comprise at least one image sensor, preferably, at least two image sensors, and the substrate comprises at least one target per image sensor for imaging by the image sensor. The delivery device is capable of translation along an x-axis and a y-axis and also is rotatable about a center axis so that the image sensors may communicate to a computer, which in turn may communicate with a mechanism such as a motor and the like that is responsible for the movement of the delivery device, to correct for deviations from the predetermined orientation for the substrate on the delivery device. Other configurations for the delivery device may also be employed.

The apparatus of the invention may also comprise an apparatus for washing certain portions of the droplet dispensing device such as the inside of the dispensing nozzles and associated chambers and the surface from which the nozzles depend. Washing is generally carried out to remove, from the aforementioned surfaces, residual reagents for synthesizing biopolymers. In one embodiment the washing apparatus or wash station comprises a plurality of receptacles for sealingly engaging each head comprising a plurality of the nozzles having the residual reagents. Once engagement has taken place, appropriate measures are applied such that residual liquid is removed from the nozzles and the nozzles are washed. Normally, the receptacles contain a wash solution and the nozzles are washed by flushing usually under pressure. This procedure involves applying pressure to the wash solution to force the wash solution into the nozzles and applying a vacuum to remove the liquid from the nozzles. Typically, the pressure involved is sufficient to force the wash solution into the nozzles and nozzle chambers without forcing the liquid beyond these points.

The wash solution may be an organic solvent or mixtures thereof or an inorganic solvent or mixtures thereof or a combination of organic solvent and inorganic solvent. The nature of the wash solution is generally governed by the nature of the biopolymer and the reagents for, and the manner of, its synthesis. Examples of organic solvents include acetonitrile, ethanol, acetone and the like. Examples of inorganic solvents include water, and so forth.

The apparatus for washing also comprises a wet wash pad for engaging a surface comprising the nozzles as well as the outer surfaces of the nozzles themselves. The wet wash pad is designed to provide a wet wiping of the surface of the nozzles and the surface from which the nozzles depend. The wet wash pad may be manufactured from any material that will accomplish the intended function. Usually, the wet wash pad is a porous material that provides some resiliency or absorbency or sponge-like quality. The wash pad may be composed of fibers, mesh, or the like. The wash pad may be constructed from materials such as, for example, paper, cellulosil sponge, polyvinylchloride, polyacrylamide, cellulose acetate, and so forth. The dimensions of the wash pad are usually determined by the dimensions of the surfaces to be washed.

The washing apparatus further comprises a dry pad for engaging the surface comprising the nozzles as well as the nozzles themselves. The dry pad may be constructed of a material that is the same as or similar to that for the wet wash pad. The dry pad is designed to remove substantially all residual liquid from the surface of the nozzles as well as the surface from which the nozzles depend (such as the face of a print head), usually by wiping. Accordingly, the dry pad should be able to remove and absorb liquid to assist in the drying process. Typically, the surfaces dried by the dry pad are at least about 90% free of, usually, at least about 95% free of, more usually, at least 99.9% free of, residual liquid. The dimensions of the dry pad are usually the same as or similar to the dimensions of the wash pad, but need only be sufficient to accomplish the dry wiping of the surface that is washed.

The washing apparatus is designed in such a manner as to repeat the engagement of the dry pad with the surface of the dispensing device comprising the nozzles. This may be carried out any number of times to ensure that all residual liquid has been removed. Usually, the dry wiping is repeated about 2 to about 6 times. The present apparatus may be equipped with a suitable device for visualizing the surface of the dispensing device that comprises the nozzles and to determine if any residual liquid remains. Such a device may be, for example, a camera, and so forth. The visualization device communicates with a computer, which in turn communicates with the mechanism for moving the wash apparatus. If the visualization device determines that residual liquid remains on the surface, the computer instructs the wash apparatus to repeat the passage of the dry pad.

It should be noted that the aforementioned washing apparatus may be employed independently for washing any dispensing device, not just the aforementioned apparatus of the invention. Furthermore, the dry wiping pad of the washing apparatus may be employed independently with any washing apparatus used for wet cleaning of surfaces of dispensing devices.

The washing apparatus also comprises a mechanism for moving the washing apparatus relative to the droplet dispensing nozzles such that the nozzles serially engage the plurality of receptacles, the wet wash pad and then the dry pad. Thus, the dispensing device usually remains stationary during the washing procedure and the washing apparatus is moved relative to the dispensing device in such a way as to accomplish the above. The washing station is usually designed to move parallel to and adjacent to the substrate mount. Usually, the wash apparatus moves transversely to the direction of movement of the dispensing device. In one approach the washing apparatus comprises a housing with a recessed compartment in which the aforementioned parts of the washing apparatus are secured. The washing apparatus has associated with it appropriate circuitry, motors and the like under computer control for automating the above process. The dimensions of the compartment are governed by the dimensions of the receptacles and the wash pad and the dry pad. The housing may be constructed from any suitable material that will provide the necessary structure and support for the components of the wash apparatus during use. Such materials include, for example, metal, plastic, composite materials, and the like.

In one embodiment the plurality of receptacles for sealingly engaging each of the heads having the residual reagents may be designed as described in U.S. Pat. No. 6,323,043, the relevant disclosure thereof being incorporated herein by reference. Briefly, the apparatus is designed to provide a backpressure of predefined value to a nozzle to allow for easy purging and cleaning of the nozzles. The disclosed apparatus is designed such that regions around and outside nozzle orifices can be cleaned while preventing cleaning fluid from entering the nozzles, by providing a positive pressure to the nozzles. The cleaning may be accomplished by positioning the surface of the dispensing device from which the nozzles depend with the orifice of the nozzles adjacent and facing the receptacles of the present washing apparatus, which are in communication with a reservoir containing the wash solution. The wash solution is applied to the nozzles using a hold off pressure to prevent the wash solution from entering a reservoir chamber of the dispensing device. The hold off pressure is sufficiently positive to prevent cleaning fluid from entering the delivery chamber of the dispensing device. The disclosed cleaning station may, for example, comprise a pad carrying cleaning fluid and the head may be exposed to the cleaning fluid by wiping at least one of the head and pad across the other. The apparatus may further include a processor which directs the positioning system to selectively position the dispensing device at any of the stations, which may also direct the pressure source to provide the required pressures when the surface comprising the nozzles is facing the corresponding stations, and which may cause the positioning system to position the wash station and the dispensing device in accordance with the above teaching.

One embodiment of the present invention is a method for washing droplet dispensing nozzles to remove residual reagents for synthesizing biopolymers, the method comprising:
(a) sealingly engaging each of the nozzles having the residual reagents therein with a receptacle containing a wash solution,
(b) flushing each of the nozzles with the wash solution,
(c) disengaging each of the nozzles from respective receptacles,
(d) engaging a surface comprising the nozzles with a wet wash pad, and
(e) engaging the surface with a dry pad.

The above method may further comprise repeating step (e) if residual wash solution is present. In the above method, step (d) may be performed by wiping the surface over the wash pad and, also, step (e) may be performed by wiping the surface over the dry pad.

One embodiment of the present invention is an apparatus for washing droplet dispensing nozzles to remove residual reagents for synthesizing biopolymers, the apparatus comprising:
(a) a plurality of receptacles for sealingly engaging each of the nozzles having the residual reagents therein, the receptacles containing a wash solution,
(b) a wet wash pad for engaging a surface comprising the nozzles, and
(c) a dry pad for engaging the surface, and
(d) a mechanism for moving the apparatus relative to the droplet dispensing nozzles such that the nozzles serially engage the plurality of receptacles, the wet wash pad and then the dry pad.

The apparatus of the present invention may also comprise a loading station for loading reagents into the dispensing device. The loading station may be positioned in the present apparatus in a manner similar to that of the wash station. Accordingly, the loading station may be placed in line with the wash station so that it moves transversely with respect to the dispensing device, which moves on the x-axis. The loading station may be of any convenient structure as long as the function of filling the dispensing device with reagents to be dispensed is accomplished. Usually, the loading station comprises a retractable cover so that the cover remains on a housing of the loading station until the loading station is employed to add reagents to the dispensing device. Various mechanisms may be employed for retracting the cover such as, for example, pulleys, belts, gears, motors, and so forth. The loading station comprises appropriate controls for controlling the temperature, humidity and the like of the components of the loading station including the reagents contained therein. The loading station also comprises appropriate circuitry and motors for controlling the movement of the loading station parallel to the x-axis.

It should be noted that the aforementioned loading apparatus may be employed independently for loading any dispensing device, not just the aforementioned apparatus of the invention.

An example of an embodiment of a suitable loading station, by way of illustration and not limitation is described in U.S. patent application Ser. No. 09/183,604, filed Oct. 30, 1998, the relevant disclosure of which is incorporated by reference. The application discloses methods and apparatus for transferring small quantities of liquids from a multiplicity of depots to a multiplicity of receptacles. The method involves transferring liquids from a plurality of wells to one or more receptacles, by displacing liquid contained in each well so that a convex meniscus swells from the opening of the well, and contacting a receptacle with the swollen meniscus to draw at least a portion of the liquid into the receptacle. The liquid transfer is effected directly from the depots to the corresponding receptacles without contact between depots and the receptacles, and without interposition of any transfer device between depots and the receptacles. And, according to the invention, the flow of the liquid into the receptacle following contact of the receptacle with the meniscus is at least initially a result of capillary interaction, and ordinarily is principally so. In one aspect liquids are transferred from a plurality of wells having openings arranged in a selected format to a plurality of receptacles arranged in a corresponding or complementary format, by displacing the liquid contained in each well so that a convex meniscus swells from the opening, and contacting the corresponding receptacle with the swollen meniscus to draw a portion of the liquid into the receptacles. Various approaches may be employed for the liquid displacing step. This step may be carried out by inwardly deforming a wall of each well to displace the liquid. In some embodiments the wall is inwardly deformed by application of mechanical or fluid pressure to the wall. In other embodiments the liquid displacing step is carried out by introducing a gas into a part of each well away from the opening. In yet other embodiments the gas is introduced through a vent in a part of the wall away from the opening, and in some embodiments the gas is passed through a gas-permeable membrane covering the vent.

In some embodiments of the above, the arrangements of the well openings and the receptacles is such that receptacles to which transfer of liquid is specified may come into contact with swollen menisci at the openings of specified wells. In some embodiments the arrangement of either the well openings or the receptacles is in a generally planar format, and the step of contacting the receptacles with the menisci is carried out by bringing the specified receptacles with the menisci at the specified well openings. On the other hand, the arrangement of the well openings and the arrangement of receptacles each is in a generally planar format, and the step of contacting the receptacles with the menisci is carried out by bringing the well openings into respective planes into generally parallel proximity.

The disclosed apparatus for transferring a plurality of liquids includes a depot member having a plurality of wells each having an inwardly deformable wall portion and an opening, in which the openings are supported in a selected format, and a member defining a plurality of receptacles in a corresponding or complementary format; means for displacing liquid contained within the wells toward and through the openings; and means for bringing well openings and receptacles into proximity. The transfer of liquid is effected by deploying the displacing means to displace the liquid in the well, causing a convex meniscus to swell outward from the opening. When a receptacle that has been brought into proximity contacts the swollen meniscus, the liquid is drawn into the receptacle. The apparatus for effecting the transfer is uncomplicated and can be made in a straightforward manner from inexpensive materials using simple tools.

For some processes, it may be advantageous to transfer a multiplicity of liquids from a multiplicity of specified wells or depots to a multiplicity of assigned or specified receptacles in a single transfer operation. Accordingly in some embodiments the well openings and the receptacles are arranged so that a multiplicity of corresponding or complementary receptacles and wall openings can be brought into proximity simultaneously, so that the receptacles contact the respective menisci at the same time. Where the well openings are arranged in a generally planar pattern, for example, liquid droplets expressed at a line of such wells may in one step be transferred into a line of receptacles that are brought into generally parallel proximity with the line of wall openings; or, liquid droplets at a planar group of such wells may in one step be transferred into a complementary group of receptacles, themselves arranged in a generally planar pattern, that are brought into generally parallel proximity with the group of well openings.

Accordingly, in some embodiments the receptacle-defining member is generally planar, and the well openings are supported in a generally planar format. In some embodiments the receptacle-defining member is an orifice plate of a print head and the receptacles are in fluid communication with reservoirs in the print head; and in some embodiments the print orifices are the receptacles. In some embodiments the wells include a deformable wall portion, and the means for displacing the liquid in the wells include means for inwardly deforming the deformable wall portion. In some embodiments the wall-deforming means includes mechanical means such as a plunger for pressing against an outer surface of the deformable wall portion; or means for applying fluid pressure (liquid or gas) at the outer surface of the deformable wall portion. In other embodiments each well includes a vent positioned away from the opening, and means for introducing a fluid (gas or liquid) through the vent and into the well, to displace the liquid in the well toward the opening. In preferred embodiments the vent is covered by a membrane that retains the liquid in the well under operating conditions, but is permeable to the fluid to be introduced through the vent into the well to displace the liquid in the well. In some embodiments the well includes a rigid wall portion in addition to the deformable wall portion, and in some embodiments the inwardly deformable wall portion and the rigid wall portion are formed of a unitary piece of material. In some embodiments the inwardly deformable wall portion includes a plastic or elastic film. In some embodiments the inwardly deformable wall portion and the support for the well openings are formed of a unitary piece of material.

The present apparatus may also comprise a mechanism and method for accurately and rapidly observing deposition of droplets of liquid on the surface of a substrate. One such mechanism and method is described in U.S. Pat. No. 6,232,072 B1, issued May 15, 2001 (Fisher). The method includes depositing droplets of fluid carrying a biopolymer or a biomonomer on a front side of a transparent substrate or support. Light is directed through the substrate from the front side, back through a substrate back side and a first set of deposited droplets on the first side to an image sensor. In this manner, the first set is “imaged”. The light may optionally pass through the substrate from the front side at a position other than the first droplet set before being reflected to pass back through the back side of the substrate and first droplet set. Particularly, the light may pass through the substrate from the first side at an angle to a normal of the first side, and pass back through the back side and first droplet set at a complementary angle to the normal. Alternatively, the light may pass through the first droplet set when passing through the substrate from the first side, before being reflected to again pass through the first droplet set. In either event, the light is optionally reflected at a position spaced from the back side. The image sensors employed in this approach are similar to those described above. Other mechanisms may be utilized and, depending on the nature of the camera, different orientations of lighting may be used. A key point with respect to the present invention is that one is able to do inspection of the surface while dispensing action is being conducted.

The directing of light in the foregoing manner may be repeated for additional sets of the deposited droplets by scanning the directed and reflected light across the first side. This can, for example, be accomplished by scanning both a light source of the directed light and the image sensor in unison across the first surface. Furthermore, the droplets may be deposited as droplet sets by a head, and multiple droplet sets may be deposited by scanning the head across the first side. Any deposited set may or may not be the same set that is later imaged by the sensor as a set. Further, the light source, image sensor and head are preferably physically interconnected and are scanned in unison across the first surface. The mirror preferably faces at least that area on the second side corresponding to that area on the first side across which droplet sets are deposited.

A mechanism or apparatus that can execute the aforementioned inspection method includes a light source, reflector, and image sensor. The apparatus includes other features that are part of the present apparatus such as the substrate mount and so forth. The mechanism may be associated with the dispensing device in that it is affixed to the same structural member as the dispensing device. In this way the inspection mechanism moves in the same manner as the dispensing device. If the inspection mechanism is not affixed in this manner, it may further include a transport system for the head, light source and image sensor, so as to move them in a manner as described, preferably including scanning in unison (with the head, light source, and image sensor being preferably physically interconnected as described above). A processor may also be provided to control the transport system as required.

A particular example of an apparatus in accordance with the present invention is described next by way of illustration and not limitation. The substrate mount is in the form of a vacuum chuck that is carried by a stacked Increment-theta stage arrangement that is attached directly to a granite base. A print head assembly is carried by an orthogonal stacked Scan-Z stage arrangement that is attached directly to a rigid granite beam off the granite base. This arrangement makes for a stiff, decoupled structure allowing for more design options. The Increment (y-axis), Scan (x-axis) and Z-axis are all mounted normal to each other to form a Cartesian positioning arrangement. The Theta axis provides rotation of the substrate about an axis parallel to the Z-axis. To achieve a parallel relationship between the substrate and the Scan-axis, two cameras are mounted on the Increment-axis for viewing targets on the substrate through access holes in the vacuum chuck. Initial calibration of the system defines the orientation of the Scan-axis relative to these cameras. In this way the substrate can be automatically oriented by direct image processing of the targets and rotating the Theta-axis to correct any rotational offset.

An important aspect of printing is the height or gap between the print head and substrate. This must be set and maintained by the Z-axis prior to printing. Determination of the gap is done using two vertical touch probes as mentioned above. One probe is mounted on the Z-axis and is used to measure the height of items, e.g., substrate, that are carried by the Increment-axis and the Theta-axis. A second probe is mounted on the Increment-axis and is used to measure and determine the height and co-planarity of the print head relative to the substrate. A third upward looking camera is positioned on the Increment-axis to assist in aligning the individual print head and vertical touch probe during initial calibration. A fourth camera mounted on the Z-axis is used for overall system calibration and also functions to locate print head service station components and a second touch probe mounted on the Increment-axis. Initial calibration of the system defines the offset between the probe pair permitting the print head gap and orientation to be set accurately prior to printing. In process, the same touch probes are used to measure and compensate for any variation in substrate thickness.

Print head loading is done directly from two microtiter trays that are supported off the Increment-axis to the left or right, usually, left, of the vacuum chuck. The trays are housed in a covered garage with humidity control to keep the reagent fluid (biopolymer fluid reagent, biomonomer fluid reagent, etc.) from evaporating. During loading the garage door is slid open to expose the top surface of the microtiter trays. Each tray consists of a rectilinear two-dimensional array of individual wells each containing a reagent fluid, which may be the same or different, usually, different. The well spacing matches that of the print head chambers. The wells incorporate a unique collapsible lining that is exposed from the bottom. Positioned below the tray and mounted directly to the granite surface is an eject pin assembly. The eject pin assembly consists of an array of blunt pins that can be positioned along two axes. Eject-X (parallel with the Scan-axis) and Eject-Z parallel to the Z-axis. Reagent fluid is transferred to the print head by aligning and translating the eject pin array upward such that it collapses the well lining, causing the fluid to rise to the top of the tray forming a droplet. The droplet contacts a receiving print head chamber nozzle and is drawn into the chamber via capillary action and some back pressure. Typically, twenty chambers are loaded in parallel. Since the print head assembly consists of sixty chambers, the eject process is stepped along the Eject-X-axis and repeated two more times to complete the load. The combination of Eject-X-axis, print head Scan-axis, and microtiter tray Increment-axis permit the eject pin array/print head chamber combination to be positioned over/under any allowable microtiter well location.

Flushing, wet wiping and dry wiping the print head is performed by a service station located on the Increment-axis. The service station is made up of three sites. The first site consists of receptors for engaging the six individual heads that make up the print head assembly. The receptors form a tight seal around the nozzles and are used for flushing various fluids into and out of the print head chambers. The second site is a wet wash of the face of the print head to remove any residual reagent fluid that may have collected on the outside face of the print head. The third site is a dry wipe of the print head to remove any residual liquid.

Real-time visual inspection of the output from the print head during printing is performed by a line scan camera, associated optics, automatic image processing software and general purpose computer. The camera assembly is attached directly to the Scan-axis and located to the right of the print head assembly. This arrangement permits the reagent fluid to be inspected immediately after being fired onto the substrate and before it evaporates. Real-time image processing reveals the location and area (volume inferred) of every fired spot permitting multipass repair for missing spots.

The aforementioned specific embodiment of an apparatus and method in accordance with the present invention achieves precision, speed and reduces system complexity. Precision is obtained using a fixed base (in this embodiment, a granite base) to support the Scan and Increment stages along with a combination of cameras and touch probes to locate the actual position of the print heads, substrate, loading and wash stations. Speed is obtained by decoupling the Scan and Increment axes so that the print head and substrate loads are properly distributed for maximizing print speed. Combining the load and wash stations as part of the Increment stage minimizes system complexity. These advantages are realized in general in the practice of the present invention in its broadest aspects.

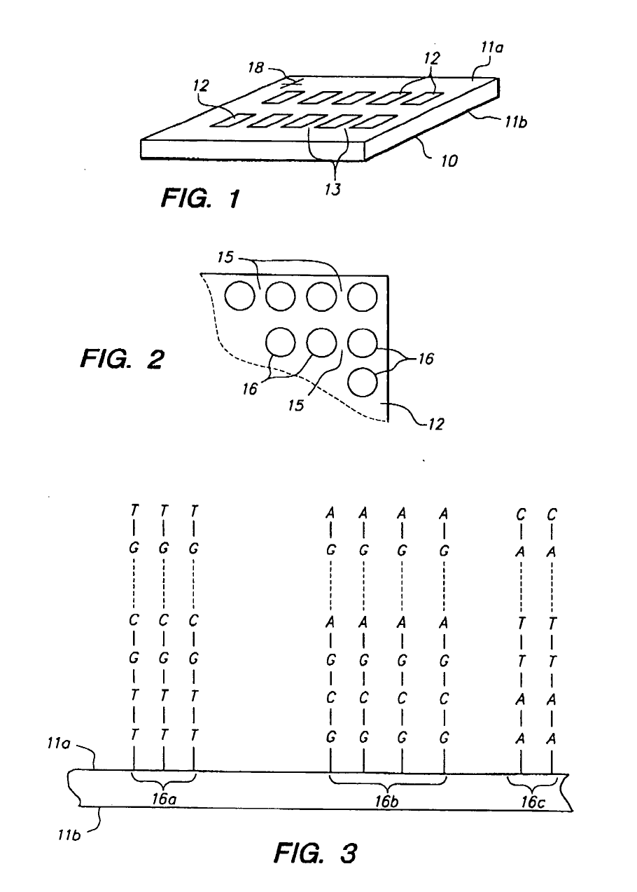

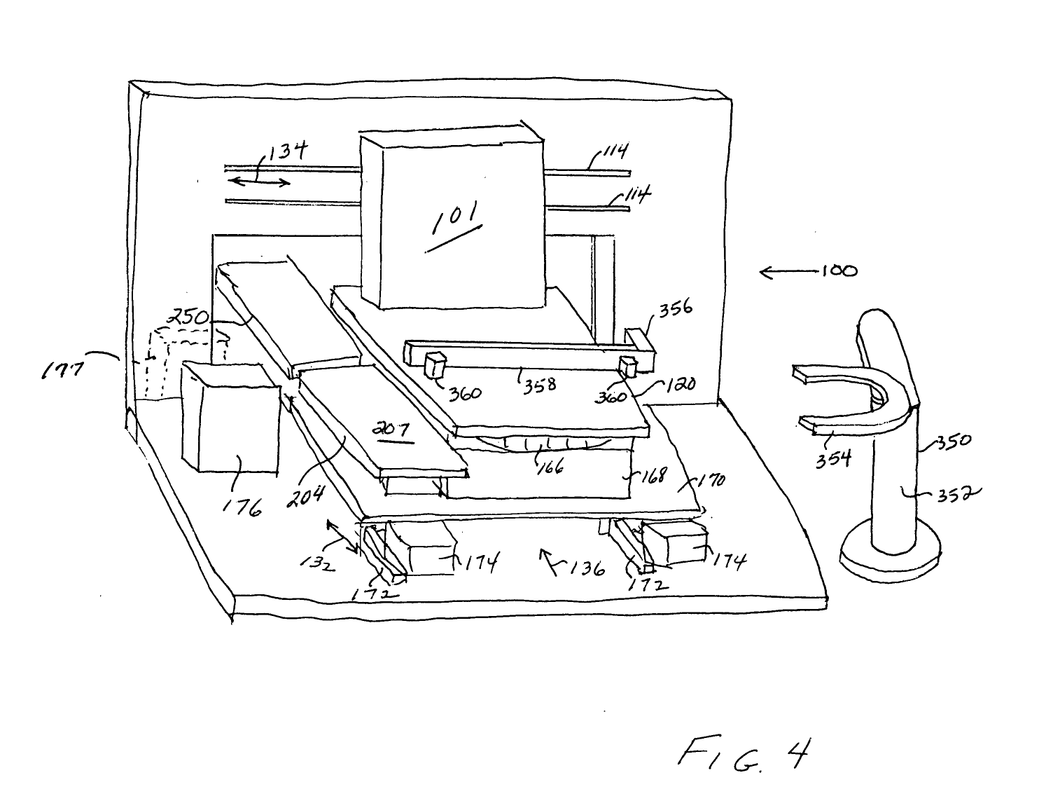

A specific embodiment of the present apparatus and method is next described in detail with reference to the accompanying drawings. As a general note, figures are not to scale and some elements of the figures may be accentuated for purposes of illustration. Also, some of the figures may not show all elements of the apparatus. Referring first to FIGS. 1-3, typically the present invention will produce multiple identical arrays 12 (only some of which are shown in FIG. 1), separated by inter-array regions 13, across the complete front surface 11a of a single transparent substrate 10. However, the arrays 12 produced on a given substrate need not be identical and some or all could be different. Each array 12 will contain multiple spots or features 16 separated by inter-feature regions 15. A typical array 12 may contain from 100 to 100,000 features. All of the features 16 may be different, or some or all could be the same. Each feature carries a predetermined moiety (such as a particular polynucleotide sequence), or a predetermined mixture of moieties (such as a mixture of particular polynucleotides). This is illustrated schematically in FIG. 3 where different regions 16 are shown as carrying different polynucleotide sequences. Arrays of FIGS. 1-3 can be manufactured by in situ or deposition methods as discussed herein.

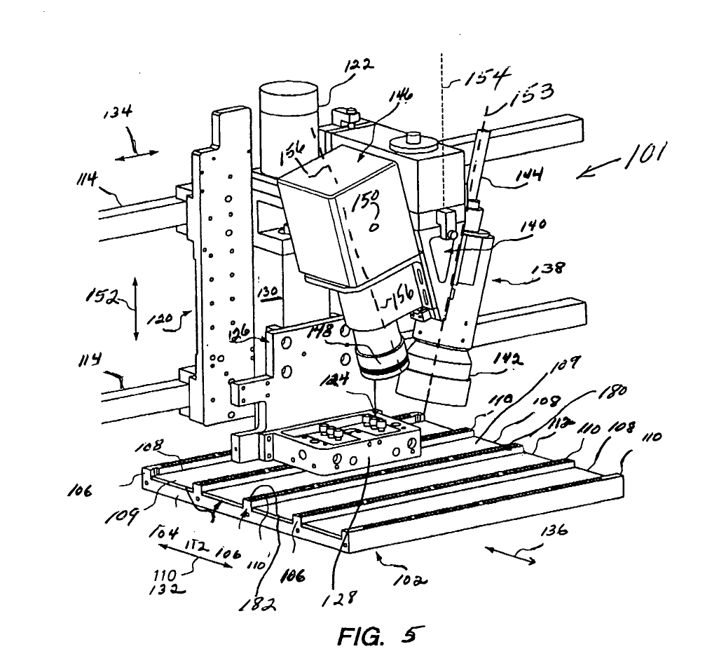

Referring to FIGS. 4 and 5, the apparatus includes camera assembly 101 and a mount for substrate 10 in the form of chuck 102. Chuck 102 is a vacuum chuck of generally rectangular configuration, and includes a bottom plate 104, and a plurality of upstanding parallel linear ribs 106 which define a series of parallel rectangular channels 108 between them. Ribs 106 have upper surfaces 110 with openings that communicate with a suitable vacuum source (not shown), such that ribs 106 can hold against, and support, a second side 11b of a mounted substrate 10. As can be seen from FIG. 6, substrate 10 has target images 14a and 14b on underside 11b. A mirror 112 is provided, in the form of multiple mirror segments 109 extending along the bottom of respective channels 108 between ribs 106, so as to be spaced from a back side 11b of a mounted substrate 10. Mirror segments 109 may be defined by a metallized reflecting layer on the back surface of a glass or other transparent substrate although front surfaced mirrors could be used if desired. In this situation it will be understood that reference to a “mirror” refers to the actual reflecting layer.

Apparatus 100 further includes two tracks 114 along which a first frame member 120 can be precisely moved by means of a motor 122 (also mounted on frame member 120) working against tracks 114 through a track drive. Tracks 114, frame member 120, motor 122 and the track drive basically act as a transporting system. Print heads 124 are provided to deposit droplets of biopolymer or biomonomer solution onto the front side 11a of a mounted substrate 10. Heads 124 are mounted to a third frame member 126 by a print head assembly 128, third frame member 126 being slidable toward and away from chuck 102 on a second frame member 130 fixedly mounted to first frame member 120.

Print heads 124 may be of a type commonly used in an ink jet type of printer and each carrying hundreds of ejectors to deposit droplets. However, it will be appreciated that drop deposition devices other than heads 124 could be used. In the case of heads 124, each ejector is in the form of an electrical resistor operating as a heating element under control of the processor (although piezoelectric elements could be used instead). Each orifice with its associated ejector and a reservoir chamber, acts as a corresponding pulse jet with the orifice acting as a nozzle. In this manner, application of a single electric pulse to an ejector causes a droplet to be dispensed from a corresponding orifice (or larger droplets could be deposited by using multiple pulses to deposit a series of smaller droplets at a given location). Certain elements of heads 124 can be adapted from parts of a commercially available thermal inkjet print head device available from Hewlett-Packard Co. as part no. HP51645A. However, other head configurations such as, for example, pin-spotting, can be used as desired.

A suitable motor and drive mechanism, which act as second transporting system, are provided inside second frame member 130 to cause such movement. Chuck 102 is mounted for movement in a direction 132 as discussed more fully hereinbelow. Thus, the first and second transporting systems act to scan heads 124 across front surface 11a of a mounted substrate 10 to deposit multiple droplets of biopolymer of biomonomer solution. Such scanning would normally be done in a row-by-row format. In the row-by-row format, heads 124 are first moved by the transport system in the direction of axis 134, which movement is coordinated by a suitably programmed processor (not shown) with firing of the pulse jets of heads 124 to deposit a row of droplets in accordance with a target array pattern. Note that by virtue of the construction of head 124 as described below, a “row” will typically include multiple lines of droplets. Substrate 10 is then moved by the transport system parallel to axis 132, specifically in the direction of arrow 136 at least the width of one row, and the process repeated. Of course, other deposition formats could be used.

A light source 138 is mounted to first frame member 120 through block 140 to direct light through substrate 10 front the front side 11a. Light source 138 includes a lens 142 and an optical fiber bundle 144, which communicates light in the visible region (substantially 400 nm to 700 nm) from a suitable source (not shown). A linescan camera 146 includes an adjustable focus lens 148 and a linear CCD or other linear sensor 150 oriented parallel to axis 132. Camera 146 (and hence sensor 150) are mounted to first frame member 120 by being mounted on a side of block 140 opposite that of light source 138. Note that the block 140 is so constructed such that an angle α between the light directed by light source 138 through mounted substrate 10 (shown by axis 153) and a normal 154 to first side 11a of mounted substrate 10, and the angle β between the light which is reflected light back through the back side 11b (shown by axis 156) and normal 154, are complementary angles (that is, of the same magnitude but in opposite directions about normal 154). The total angle (α+β) should be kept as small as possible (such as less than 50° or even less than 40°), limited only by the physical size of the components, to provide a compact arrangement. Furthermore, keeping the angle small limits the distance at which mirror 112 needs to extend beyond the array being formed. Note also that the direction of the light from light source 138 (as illustrated by axis 153) and to sensor 150, lie on a common plane, which is oriented in the direction of channels 108 (specifically, by being parallel to those channels 108).

Referring to FIGS. 4, 5 and 7, apparatus 100 further comprises two cameras 158 and 160, which are each respectively mounted on frame members 162 and 164. Vacuum chuck 102 is mounted on theta stage 166, which in turn is mounted on increment stage 168. Frame members 162 and 164 and increment stage 168 are secured to plate member 170, which is slidably mounted to move in direction 132. Apparatus 100 further comprises motor assembly 177, which drives, among others, the Increment axis and the Scan axis. The apparatus further includes two tracks 172 along which frame members 174 can be precisely moved by means of motor assembly 177 working against tracks 172 through a track drive. Tracks 172, frame members 174, motor 177 and the track drive act as a transporting system for moving the vacuum chuck in a direction transverse to the direction of movement of heads 124 so that droplets of biopolymer or biomonomer solution are deposited onto the front side 11a of a mounted substrate 10 as discussed above. Apparatus 100 further comprises motor assembly 176, which drives, block 227. Motor assembly 176 and motor assembly 177 each comprise appropriate motors, pulleys, drive belts and the like normally associated with such assemblies. Motor assembly 176 and motor assembly 177 are controlled by a suitable processor and computer for controlling the timing and movement of the substrate mount, dispensing device, and so forth.

Vacuum chuck 102 comprises two openings 180 and 182, which are set in vacuum chuck 102 corresponding to the viewing line of cameras 158 and 160, respectively. As discussed above, the openings are of a character that permits the camera to image target images 14a and 14b, respectively, on side 11b of substrate 10.

Referring to FIGS. 4 and 7, apparatus 100 also comprises delivery device 350 with support 352 and moveable arm 354. Delivery device optical system 356 includes support member 358 and cameras 360.

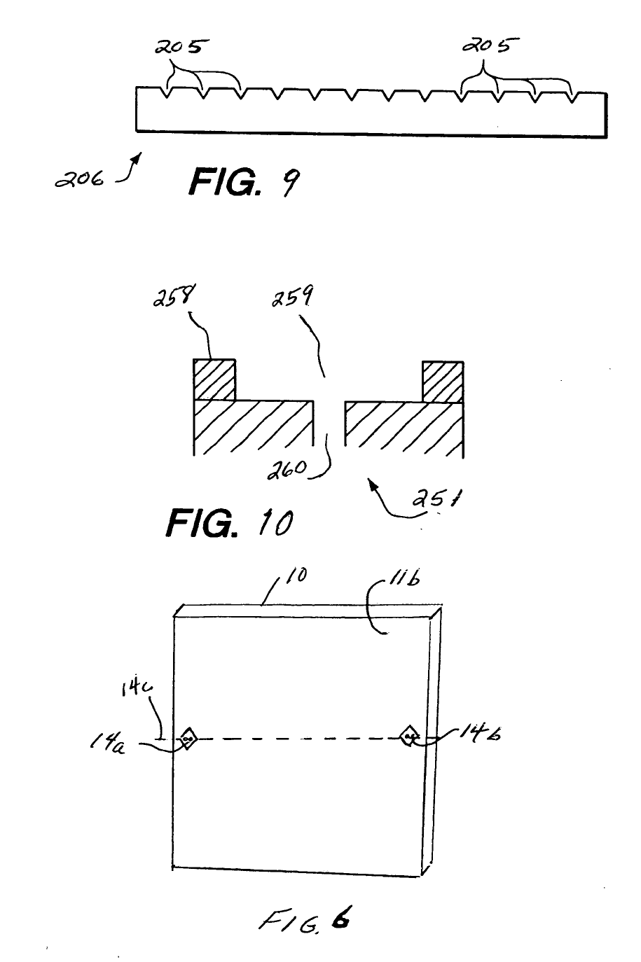

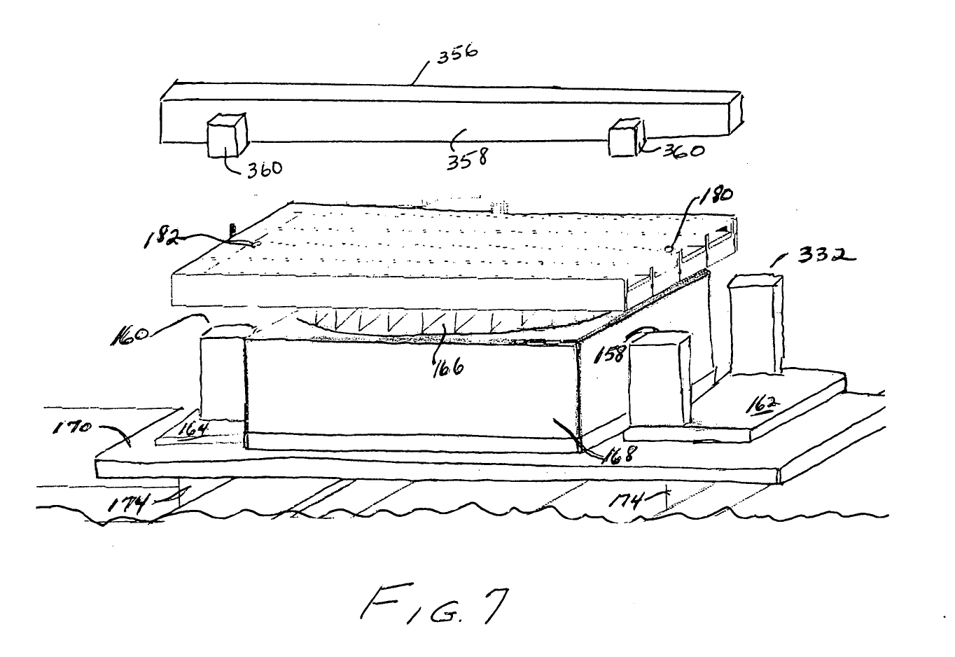

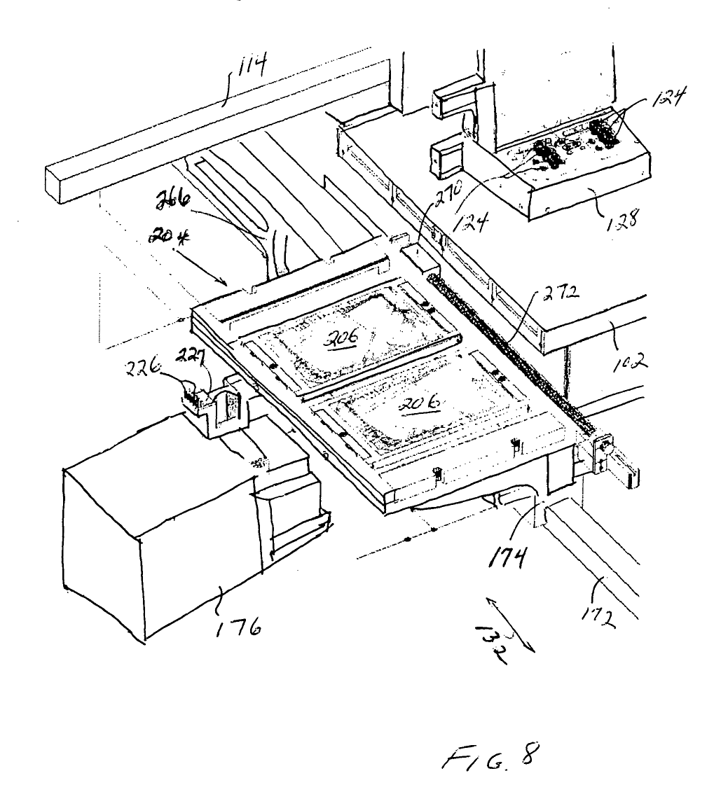

Referring to FIG. 8, apparatus 100 also comprises loading station 204 that comprises two loading blocks 206, which can be of any construction with regions that can retain small volumes of different fluids for loading into heads 124. For example, it may be a glass surface with different hydrophobic and hydrophilic regions to retain different drops thereon in the hydrophilic regions. Alternatively, the flexible microtiter plate described in U.S. patent application entitled “Method and Apparatus for Liquid Transfer,” Ser. No. 09/183,604 could be used. Referring to FIGS. 8-10, loading block 206 has an upper surface with small notches 205 to assist in retaining multiple individual drops of a biomonomer or biopolymer fluid on that surface. The number of notches 205 or other regions for retaining drops of different fluids, is at least equal to (and can be greater than) the number of reservoir chambers in print heads 124, and are spaced to align with orifices in heads 124. Even where the number of such fluid retaining regions is less than the number of orifices, all delivery chambers communicating with one another (through a reservoir chamber) can still be filled in this embodiment as described in the aforementioned patent application. Defined load pressure values are employed wherein fluid that has entered a reservoir chamber through one orifice can still be drawn by capillary pressure into other delivery chambers communicating with the same reservoir chamber.

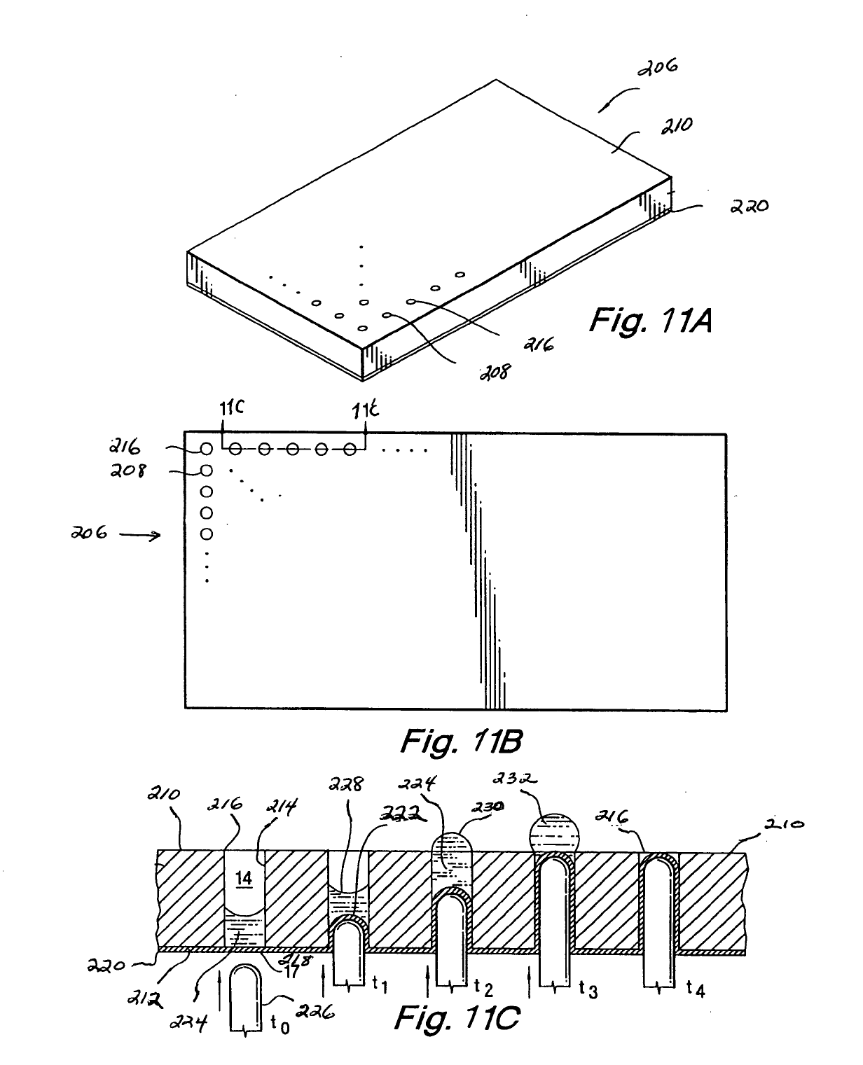

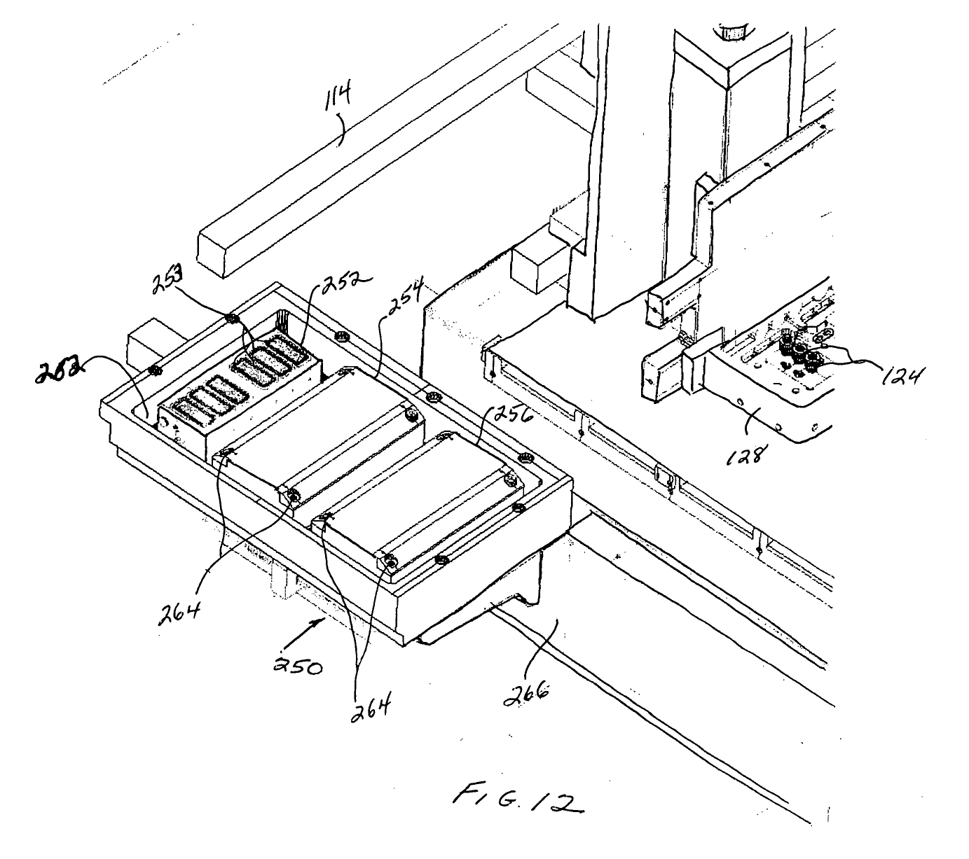

In a related design (referring to FIGS. 11A, 11B and 11C, loading blocks 206 each comprise a plurality of depots 208, from which liquids are to be transferred according to the invention to reservoirs arranged in a corresponding pattern. Loading station 204 is affixed to frame member 266, which is fixedly attached to plate member 170. This normally involves movement of frame members 174 along tracks 172 as discussed above to move plate 170. Motor system 177 can be operated to move loading station 204 in either direction along 132 so that loading station 204 may be moved into position under print head assembly 128 to load the print reservoirs with reagent fluid. Print head assembly 128 is moved into position over loading station 204 as described above. As is evident, print head assembly 128 and loading station 204 move transversely to one another. Loading station 204 generally also comprises a retractable cover 207. In the embodiment illustrated herein, depots 208 are arranged as wells of a standard 16×24 microtiter plate (not all the wells are drawn in FIGS. 11A-11C). As may be understood more clearly by reference to FIG. 11C, depot blocks 206 have a generally planar surfaces 210 and 212, and each generally cylindrical depot 208 is defined by a generally cylindrical rigid wall 214 passing from surface 210 through to surface 212 of depot blocks 206 and having a generally circular opening 216 at surface 210 and a generally circular opening 218 at surface 212. Depot block surface 212 is covered with a plastic or elastic film 220, which is sealed to surface 212 at least at the margins of circular openings 218. Accordingly, the circular depot openings 216 are arranged in a generally planar pattern, and the portions of the elastic film 220 that cover the circular openings 218 form the deformable wall portions 222 of the depots.

The embodiment of FIG. 11A-11C is employed in the apparatus of the invention as follows. Referring to FIG. 11C, which is a composite showing a time course (t₀ through t₄) from left to right, a quantity of liquid 224 is held in each depot. In practice, the liquid in each depot may have a specified character, or the liquid in each depot may contain a specified biomolecule or reagent or analyte, or a specified mixture of biomolecules or reagents or analytes. The liquids may simply be stored in the depots, or they may have been prepared in the depots at least in part. The depots may be entirely filled with the liquids or, as illustrated in FIG. 11C (t₀), they may be only partly full. Transfer from a depot is initiated (t₁) by applying a force against the deformable wall portion 222, deforming it inward and displacing the liquid 224 within the depot. The wall may be deformed by any of a variety of means for applying force; one such means, shown by way of illustration in FIG. 11C, is to press a plunger 226 inwardly against the outer surface of the deformable wall portion. The progressively increasing inward deformation of the wall causes the meniscus 228 of the liquid to rise toward (t₁) and through (t₂) the opening 216, forming a convex meniscus 230. As the liquid 224 is further displaced (t₃) the convex meniscus 230 rises and swells as a droplet 232 of the liquid is held by surface interaction away from the opening 216 and the depot block surface 210. The transfer is completed (t₄) by contacting the swollen convex meniscus 230 with a reagent reservoir of print head 124. Surface interaction of the reservoir with the liquid results in movement of the droplet of fluid 232 away from the depot block surface 210 and the opening 216 and into the reservoir. A plurality of plungers are depicted in FIG. 8 on U-shaped block 227, which is adapted with appropriate motors as part of motor systems 176 and 177 to move block 227 into position and to move plungers 226 in an upward direction toward the underside 212 of loading blocks 206 of loading station 204.

Loading station 204 comprises retractable cover 207, which is fixedly attached to member 270, which is part of actuator mechanism 272. Retractable cover 207 is moved by the movement of mechanism 272 that is designed with appropriate motors to move retractable cover 207. The operation of mechanism 272 may be more fully understood with reference to FIG. 8.

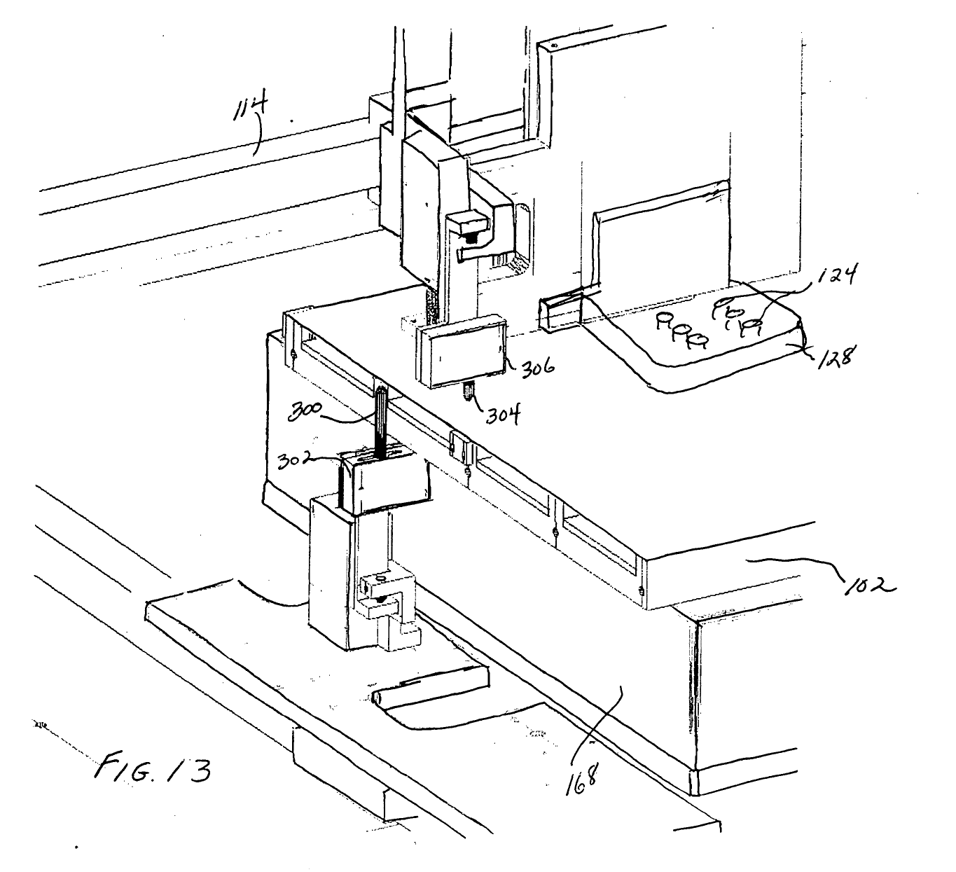

Referring to FIGS. 4, 10 and 12, wash station 250 is also affixed to frame member 266 adjacent, and to the rear of, loading station 204. It should be noted that loading station 250, which normally resides in front of wash station 250, is not shown in FIG. 12 for purposes of more clearly depicting the components of wash station 250. Motor assembly 177 can be operated to move wash station 250 in either direction along 132 so that wash station 250 may be moved into position under print head assembly 128 to flush the print heads, wash the print head surfaces and subsequently dry the print head surfaces. Print head assembly 128 is moved into position over wash station 250 as described above. As is evident, print head assembly 128 and wash station 250 move transversely to one another. Suitable processors and computers are employed for this purpose.

Referring to FIGS. 10 and 12, wash station 250 comprises a flushing station 252, a wash pad 254 and a dry pad 256. Flushing station 252 comprises a plurality of receptacles 253 for sealingly engaging each of the nozzles of print heads 124. Each of receptacles 251 has an upper surface defined by a generally rectangular urethane gasket 258 and a region 259 interior of gasket 258. Interior region 259 communicates with a vacuum line 260. A vacuum source (not shown) communicates through vacuum line 260 and an electrically controlled valve (not shown), which is controlled by a processor through a control line (both not shown). Vacuum source may include a suitable vacuum supply (such as a pump) as well as a trap. Gasket 258 is dimensioned such that a periphery of a front face of a dispensing head 124 can sealingly engage against upper surface 258 with interior region 259 aligned and communicating with the two rows of orifices in head 124. In this manner, the orifices can be placed in communication with vacuum line 260 so that, during a purging step (described further below) vacuum from line 260 can pull fluid out of head 124 through the orifices. The processor may be a general purpose microprocessor suitably programmed to execute all of the steps required by the present invention, or any hardware or software combination which will perform the required functions.

Wash pad 254 is an upwardly facing pad that can be saturated with a suitable cleaning fluid or wash solution. The composition of wash pad 254 and the nature of the wash solution are discussed above. After print heads 124 of print head assembly 128 have been flushed at flushing station 252, the face of print head assembly 128, i.e., the surface of the print head carrying the printing nozzles, is contacted with the wash pad. Print head assembly 128 having been moved into position over wash station 250, wash station 250 is moved into position so that print head assembly 128 may be lowered into contact with wash pad 254. Movement of wash pad 254 is accomplished by the respective components of motor assembly 177. Usually, print head assembly 128 is lowered into contact with wash pad 154 and wash pad 154 is moved transversely to print head assembly 128. In this way the face of print head assembly 128 is wiped with the wet wash pad.

Dry pad 256 is also an upwardly facing pad that is dry so that it may remove residual liquid from the face of print head assembly 128. Both wash pad 254 and dry pad 256 are secured in the interior chamber 262 of wash station 250 by means of retainers 264. The composition of the dry pad 256 may be the same as, or different from, that of wash pad 254. In operation, after the face of print head assembly 128 has been washed at wash pad 254, the face of print head assembly 128 is contacted with dry pad 256. As with wash pad 254, usually, print head assembly 128 is lowered into contact with dry pad 256 as dry pad 256 is moved transversely with respect to the direction of movement of print head assembly 128. In this way the face of print head assembly 128 is wiped with the dry pad. As explained above, this process is repeated until the face of print head assembly 128 as well as the exterior surfaces of print heads 124 are dry. As explained above, appropriate viewing sensors may be employed to view the face of print head assembly 128.

Apparatus 100 also comprises a touch system. Referring to FIG. 13, an upwardly pointing touch probe 300 is fixedly attached to block 302 and a downwardly pointing touch probe 304 is fixedly attached to sensor mechanism 306. Camera 332 (FIG. 7) is also mounted on frame member 162 and is adapted to view one or more target images (not shown) on the bottom side of print head assembly 128.

The above apparatus is used to fabricate an array in the following manner. It will be understood that all of the operations particularly following mounting of the substrate 10, can be controlled by a suitably programmed processor, such as a programmed general purpose processor or any hardware/software equivalent. The operations may be carried out employing suitable motor assemblies for moving the various components of the apparatus. An initial set up of the apparatus is carried out. This set up involves the touch system comprising touch probes 300 and 304 and camera 160 views probe 304.

After the initial set up and calibration of the apparatus, it may be used to synthesize arrays on surface 11a of substrate 10. It will be assumed that the heads have already been loaded with one or more biopolymer or biomonomer solutions. Substrate 10 may be mounted on vacuum chuck 20 by placing it with its back side 11b in contact with upper surfaces 110 of ribs 106. A vacuum is applied through ribs 106 to the openings so as to firmly retain second side 11b in position supported against ribs 106. The substrate mounting operation is then complete. After substrate 10 is moved into position on vacuum chuck 102, cameras 158 and 160 observe side 11b of substrate 10 through openings 180 and 182 in vacuum chuck 102. Cameras 158 and 160 observe the location of the target images 14a and 14b, the computer sends instructions to theta stage 166 to rotate vacuum chuck 102 to align the substrate to the scan axis. This process is repeated until the cameras view the target images at the correct locations. Once this occurs, the computer sends instructions for the printing process to proceed. The phrase “correct location” or “correct position” means that the scan axis of the print heads is substantially parallel to line 14c (FIG. 6), that is the imaginary line through the center of targets 14a and 14b.

Either simultaneously with, or subsequently to, the above, camera 332 examines print head assembly 128 for the target image thereon (not shown). An adjustment is made to the alignment of print head assembly 128 based on the information sent from camera 332 to the central computer, which instructs the adjustment of the print head assembly 128 to maintain a correct or parallel relationship between printing head assembly 128 and surface 11a of substrate 10.

Lens 148 is adjusted to focus on the front surface 1a of mounted substrate 10. First, print head assembly 128 is moved away from vacuum chuck 102 by causing support member 126 to slide upward on support member 130. Note that the above described construction allows print head assembly 128 to be adjusted toward or away from chuck 102 (and hence first side 11a of substrate 10) independently of movement of sensor 150. That is, camera 146 will not move during such height adjustment of print head assembly 128, and thus the focus of camera 146 is not affected by such height adjustment. Print head assembly 128 may then have its height readjusted to a suitable distance from first side 11a for dispensing fluid droplets as described above.

The processor then causes a motor assembly to move print head assembly 128 to scan across the front side 11a of substrate 10 in unison with light source 138 and camera 146, in a row by row format as described above. Simultaneously, the processor activates the pulse jets in print heads 124 in a sequence to dispense multiple droplets in co-ordination with relative movement of the head and substrate, in accordance with the target array pattern. Light source 138 and sensor 150 are positioned such that sensor 150 images a set of droplets (referenced as a “first set”), specifically a line of droplets, forming part of at least one row deposited as part or all of one or more previously deposited rows. As the scan continues along a row, sensor 150 images droplets and provides data to the processor for analysis as to droplet characteristics (for example, any one or more of whether a droplet is present, its location, or its size). The results of the analysis may be compared with the expected characteristic based on the target array pattern, and used to identify array errors, stop and/or correct the fabrication process for subsequent arrays or substrates, or be communicated to a remote or local user of the array results (either as hardcopy printed instruction, or electronically). Alternatively, the droplets observed can be part of a test print, and the results used to more carefully set up the apparatus for depositing actual biopolymer arrays.

Substrate mount 102 is moved incrementally along axis 136 by means of motor assembly 177 to present unprinted areas of surface 11a for deposition of droplets of reagent fluid. In this way the computer directs and controls the movement of substrate mount 102 along tracks 172.

When reagent fluid is to be loaded into print heads 124, print head assembly 128 is moved into position over loading station 204. Movement of print head assembly 128 is directed by the computer, which instructs a motor assembly to move print head assembly 128 along tracks 114 along axis 134. Retractable cover 207 is moved by the movement of mechanism 272 that is designed with an appropriate actuator such as, for example, a motor, air cylinder, and the like, to move retractable cover 207. Upon instruction from the computer, print head assembly 128 is then lowered by causing support member 126 to slide downward on support member 130. As mentioned above, transfer from a depot is initiated by applying a force against the deformable wall portion 222, deforming it inward and displacing the liquid 224 within the depot. To this end, plunger 226 is pressed inwardly against the outer surface of the deformable wall portion. The progressively increasing inward deformation of the wall causes the meniscus 228 of the liquid to rise toward (t₁) and through (t₂) the opening 216, forming a convex meniscus 230. As the liquid 224 is further displaced (t₃) the convex meniscus 230 rises and swells as a droplet 232 of the liquid is held by surface interaction away from the opening 216 and the depot block surface 210. The transfer is completed by contacting the swollen convex meniscus 230 with a reagent reservoir of a print head 124. As also mentioned above, block 227, which comprises a plurality of plungers 226, is adapted with appropriate motors as part of motor system 176 to move block 227 into position and to move plungers 226 in an upward direction toward the underside 212 of loading blocks 206 of loading station 204.

As mentioned above wash station 250 is employed at desired intervals to flush print heads 124 and associated chambers, to wash the exterior surfaces of print head assembly 128 at least where the print heads are located, and to dry such exterior surfaces. As described above for the loading station, movement of print head assembly 128 is directed by the computer, which instructs a motor assembly to move print head assembly 128 along tracks 114 on axis 134. Upon instruction from the computer, print head assembly 128 is then lowered to contact with receptacles 253 of flushing station 252 by causing support member 126 to slide downward on support member 130. Motor assembly 177 is operated to move wash station 250 in either direction along axis 132 so that wash station 250 may be moved into position under print head assembly 128 to flush the print heads. Suitable processors and computers are employed for this purpose. A vacuum source (not shown) communicates through vacuum line 260 and an electrically controlled valve (not shown), which is controlled by a processor through a control line (both not shown). Vacuum source may include a suitable vacuum supply (such as a pump) as well as a trap. As mentioned earlier, receptacles 252 sealingly engage print heads 124. In this manner, orifices can be placed in communication with vacuum line 260 so that, during a purging step (described further below) vacuum from line 260 can pull fluid out of head 124 through the orifices.

After print heads 124 have been flushed at flushing station 252, the face of print head assembly 128, i.e., the surface of the print head carrying the printing nozzles, is contacted with the wash pad. To this end both print head assembly 128 and wash station 250 are moved into position so that print head assembly 128 may be lowered into contact with wash pad 254. Movement of print head assembly 128 and wash pad 254 is accomplished by the respective components of motor assembly 177. Wash pad 154 is moved transversely to print head assembly 128. In this way the face of print head assembly 128 is wiped with the wet wash pad.

After the face of print head assembly 128 has been washed at wash pad 254, the face of print head assembly 128 is contacted with the dry pad. Dry pad 256 is moved transversely with respect to the direction of movement of print head assembly 128. In this way the face of print head assembly 128 is wiped with the dry pad. As explained above, this process is repeated until the face of print head assembly 128 is dry. As explained above, appropriate viewing sensors may be employed to view the face of print head assembly 128.

As mentioned above, the apparatus and the methods in accordance with the present invention may be automated. To this end the apparatus of the invention further comprises appropriate motors and electrical and mechanical architecture and electrical connections, wiring and devices such as timers, clocks, computers and so forth for operating the various elements of the apparatus. Such architecture is familiar to those skilled in the art and will not be discussed in more detail herein.

To assist in the automation of the present process, the functions and methods may be carried out under computer control, that is, with the aid of a computer. For example, an IBM® compatible personal computer (PC) may be utilized. The computer is driven by software specific to the methods described herein. Software that may be used to carry out the methods may be, for example, Microsoft Excel or Microsoft Access and the like, suitably extended via user-written functions and templates, and linked when necessary to stand-alone programs that perform other functions.

As indicated above, the present apparatus and methods may be employed in the preparation of substrates having a plurality of chemical compounds in the form of an array on the surface of such substrates. The chemical compounds may be deposited on the surface of the substrate as fully formed moieties. On the other hand, the chemical compounds may be synthesized in situ in a series of steps such as, for example, the addition of building blocks, which are chemical components of the chemical compound. Examples of such building blocks are those found in the synthesis of polymers. The invention has particular application to chemical compounds that are biopolymers such as polynucleotides, for example, oligonucleotides.

Preferred materials for the substrate itself are those that provide physical support for the chemical compounds that are deposited on the surface or synthesized on the surface in situ from subunits. The materials should be of such a composition that they endure the conditions of a deposition process and/or an in situ synthesis and of any subsequent treatment or handling or processing that may be encountered in the use of the particular array.

Typically, the substrate material is transparent. By “transparent” is meant that the substrate material permits signal from features on the surface of the substrate to pass therethrough without substantial attenuation and also permits any interrogating radiation to pass therethrough without substantial attenuation. By “without substantial attenuation” may include, for example, without a loss of more than 40% or more preferably without a loss of more than 30%, 20% or 10%, of signal. The interrogating radiation and signal may for example be visible, ultraviolet or infrared light. In certain embodiments, such as for example where production of binding pair arrays for use in research and related applications is desired, the materials from which the substrate may be fabricated should ideally exhibit a low level of non-specific binding during hybridization events.

The materials may be naturally occurring or synthetic or modified naturally occurring. Suitable rigid substrates may include glass, which term is used to include silica, and include, for example, glass such as glass available as Bioglass, and suitable plastics. Should a front array location be used, additional rigid, non-transparent materials may be considered, such as silicon, mirrored surfaces, laminates, ceramics, opaque plastics, such as, for example, polymers such as, e.g., poly (vinyl chloride), polyacrylamide, polyacrylate, polyethylene, polypropylene, poly(4-methylbutene), polystyrene, polymethacrylate, poly(ethylene terephthalate), nylon, poly(vinyl butyrate), etc., either used by themselves or in conjunction with other materials. The surface of the substrate is usually the outer portion of a substrate.

The surface of the material onto which the chemical compounds are deposited or formed may be smooth or substantially planar, or have irregularities, such as depressions or elevations. The surface may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyethylene amines, polyarylene sulfides, polysiloxanes, polyimides, polyacetates, and the like, where the polymers may be hetero- or homo-polymeric, and may or may not have separate functional moieties attached thereto (for example, conjugated). Various further modifications to the particular embodiments described above are, of course, possible. Accordingly, the present invention is not limited to the particular embodiments described in detail above.

The material used for an array support or substrate may take any of a variety of configurations ranging from simple to complex. Usually, the material is relatively planar such as, for example, a slide. In many embodiments, the material is shaped generally as a rectangular solid. As mentioned above, multiple arrays of chemical compounds may be synthesized on a sheet, which is then diced, i.e., cut by breaking along score lines, into single array substrates.

Typically, the substrate has a length in the range about 5 mm to 100 cm, usually about 10 mm to 25 cm, more usually about 10 mm to 15 cm, and a width in the range about 4 mm to 25 cm, usually about 4 mm to 10 cm and more usually about 5 mm to 5 cm. The substrate may have a thickness of less than 1 cm, or even less than 5 mm, 2 mm, 1 mm, or in some embodiments even less than 0.5 mm or 0.2 mm. The thickness of the substrate is about 0.01 mm to 5.0 mm, usually from about 0.1 mm to 2 mm and more usually from about 0.2 to 1 mm. The substrate is usually cut into individual test pieces, which may be the size of a standard size microscope slide, usually about 3 inches in length and 1 inch in width.

The invention has particular application to substrates bearing oligomers or polymers. The oligomer or polymer is a chemical entity that contains a plurality of monomers. It is generally accepted that the term “oligomers” is used to refer to a species of polymers. The terms “oligomer” and “polymer” may be used interchangeably herein. Polymers usually comprise at least two monomers. Oligomers generally comprise about 6 to about 20,000 monomers, preferably, about 10 to about 10,000, more preferably about 15 to about 4,000 monomers. Examples of polymers include polydeoxyribonucleotides, polyribonucleotides, other polynucleotides that are C-glycosides of a purine or pyrimidine base, or other modified polynucleotides, polypeptides, polysaccharides, and other chemical entities that contain repeating units of like chemical structure. Exemplary of oligomers are oligonucleotides and peptides.

A monomer is a chemical entity that can be covalently linked to one or more other such entities to form an oligomer or polymer. Examples of monomers include nucleotides, amino acids, saccharides, peptoids, and the like and subunits comprising nucleotides, amino acids, saccharides, peptoids and the like. The subunits may comprise all of the same component such as, for example, all of the same nucleotide or amino acid, or the subunit may comprise different components such as, for example, different nucleotides or different amino acids. The subunits may comprise about 2 to about 2000, or about 5 to about 200, monomer units. In general, the monomers have first and second sites (e.g., C-termini and N-termini, or 5′ and 3′ sites) suitable for binding of other like monomers by means of standard chemical reactions (e.g., condensation, nucleophilic displacement of a leaving group, or the like), and a diverse element that distinguishes a particular monomer from a different monomer of the same type (e.g., an amino acid side chain, a nucleotide base, etc.). The initial substrate-bound, or support-bound, monomer is generally used as a building block in a multi-step synthesis procedure to form a complete ligand, such as in the synthesis of oligonucleotides, oligopeptides, oligosaccharides, etc. and the like.

A biomonomer references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A biopolymer is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions.

Polynucleotides are compounds or compositions that are polymeric nucleotides or nucleic acid polymers. The polynucleotide may be a natural compound or a synthetic compound. Polynucleotides include oligonucleotides and are comprised of natural nucleotides such as ribonucleotides and deoxyribonucleotides and their derivatives although unnatural nucleotide mimetics such as 2′-modified nucleosides, peptide nucleic acids and oligomeric nucleoside phosphonates are also used. The polynucleotide can have from about 2 to 5,000,000 or more nucleotides. Usually, the oligonucleotides are at least about 2 nucleotides, usually, about 5 to about 100 nucleotides, more usually, about 10 to about 50 nucleotides, and may be about 15 to about 30 nucleotides, in length. Polynucleotides include single or multiple stranded configurations, where one or more of the strands may or may not be completely aligned with another.

A nucleotide refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides.

The nature of the support or substrate to which a plurality of chemical compounds is attached is discussed above. The substrate can be hydrophilic or capable of being rendered hydrophilic or it may be hydrophobic. The substrate is usually glass such as flat glass whose surface has been chemically activated for binding thereto or synthesis thereon, glass available as Bioglass and the like. The surface of a substrate is normally treated to create a primed or functionalized surface, that is, a surface that is able to support the attachment of a fully formed chemical compound or the synthetic steps involved in the production of the chemical compound on the surface of the substrate. Functionalization relates to modification of the surface of a substrate to provide a plurality of functional groups on the substrate surface. By the term “functionalized surface” is meant a substrate surface that has been modified so that a plurality of functional groups are present thereon usually at discrete sites on the surface. The manner of treatment is dependent on the nature of the chemical compound to be synthesized and on the nature of the substrate surface. In one approach a reactive hydrophilic site or reactive hydrophilic group is introduced onto the surface of the substrate. Such hydrophilic moieties can be used as the starting point in a synthetic organic process.

In one embodiment, the surface of the substrate, such as a glass substrate, is siliceous, i.e., the surface comprises silicon oxide groups, either present in the natural state, e.g., glass, silica, silicon with an oxide layer, etc., or introduced by techniques well known in the art. One technique for introducing siloxyl groups onto the surface involves reactive hydrophilic moieties on the surface. These moieties are typically epoxide groups, carboxyl groups, thiol groups, and/or substituted or unsubstituted amino groups as well as a functionality that may be used to introduce such a group such as, for example, an olefin that may be converted to a hydroxyl group by means well known in the art. One approach is disclosed in U.S. Pat. No. 5,474,796 (Brennan), the relevant portions of which are incorporated herein by reference. A siliceous surface may be used to form silyl linkages, i.e., linkages that involve silicon atoms. Usually, the silyl linkage involves a silicon-oxygen bond, a silicon-halogen bond, a silicon-nitrogen bond, or a silicon-carbon bond.

Another method for attachment is described in U.S. Pat. No. 6,219,674 (Fulcrand, et al.). A surface is employed that comprises a linking group consisting of a first portion comprising a hydrocarbon chain, optionally substituted, and a second portion comprising an alkylene oxide or an alkylene imine wherein the alkylene is optionally substituted. One end of the first portion is attached to the surface and one end of the second portion is attached to the other end of the first portion chain by means of an amine or an oxy functionality. The second portion terminates in an amine or a hydroxy functionality. The surface is reacted with the substance to be immobilized under conditions for attachment of the substance to the surface by means of the linking group.

Another method for attachment is described in U.S. Pat. No. 6,258,454 (Lefkowitz, et al.). A solid substrate having hydrophilic moieties on its surface is treated with a derivatizing composition containing a mixture of silanes. A first silane provides the desired reduction in surface energy, while the second silane enables functionalization with molecular moieties of interest, such as small molecules, initial monomers to be used in the solid phase synthesis of oligomers, or intact oligomers. Molecular moieties of interest may be attached through cleavable sites.

A procedure for the derivatization of a metal oxide surface uses an aminoalkyl silane derivative, e.g., trialkoxy 3-aminopropylsilane such as aminopropyltriethoxy silane (APS), 4-aminobutyltrimethoxysilane, 4-aminobutyltriethoxysilane, 2-aminoethyltriethoxysilane, and the like. APS reacts readily with the oxide and/or siloxyl groups on metal and silicon surfaces. APS provides primary amine groups that may be used to carry out the present methods. Such a derivatization procedure is described in EP 0 173 356 B1, the relevant portions of which are incorporated herein by reference. Other methods for treating the surface of a substrate will be suggested to those skilled in the art in view of the teaching herein.

The devices and methods of the present invention are particularly useful for the preparation of substrates with array areas with array assemblies of biopolymers. An array includes any one-, two- or three-dimensional arrangement of addressable regions bearing a particular biopolymer such as polynucleotides, associated with that region. An array is addressable in that it has multiple regions of different moieties, for example, different polynucleotide sequences, such that a region or feature or spot of the array at a particular predetermined location or address on the array can detect a particular target molecule or class of target molecules although a feature may incidentally detect non-target molecules of that feature.

An array assembly on the surface of a substrate refers to one or more arrays disposed along a surface of an individual substrate and separated by inter-array areas. Normally, the surface of the substrate opposite the surface with the arrays (opposing surface) does not carry any arrays. The arrays can be designed for testing against any type of sample, whether a trial sample, a reference sample, a combination of the foregoing, or a known mixture of components such as polynucleotides, proteins, polysaccharides and the like (in which case the arrays may be composed of features carrying unknown sequences to be evaluated). The surface of the substrate may carry at least one, two, four, or at least ten, arrays. Depending upon intended use, any or all of the arrays may be the same or different from one another and each may contain multiple spots or features of chemical compounds such as, e.g., biopolymers in the form of polynucleotides or other biopolymer. A typical array may contain more than ten, more than one hundred, more than one thousand or ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges.

Any of a variety of geometries of arrays on a substrate may be used. As mentioned above, an individual substrate may contain a single array or multiple arrays. Features of the array may be arranged in rectilinear rows and columns. This is particularly attractive for single arrays on a substrate. When multiple arrays are present, such arrays can be arranged, for example, in a sequence of curvilinear rows across the substrate surface (for instance, a sequence of concentric circles or semi-circles of spots), and the like. Similarly, the pattern of features may be varied from the rectilinear rows and columns of spots to include, for example, a sequence of curvilinear rows across the substrate surface (for example, a sequence of concentric circles or semi-circles of spots), and the like. The configuration of the arrays and their features may be selected according to manufacturing, handling, and use considerations.

Each feature, or element, within the molecular array is defined to be a small, regularly shaped region of the surface of the substrate. The features are arranged in a predetermined manner. Each feature of an array usually carries a predetermined chemical compound or mixtures thereof. Each feature within the molecular array may contain a different molecular species, and the molecular species within a given feature may differ from the molecular species within the remaining features of the molecular array. Some or all of the features may be of different compositions. Each array may contain multiple spots or features and each array may be separated by spaces or areas. It will also be appreciated that there need not be any space separating arrays from one another. Interarray areas and interfeature areas are usually present but are not essential. As with the border areas discussed above, these interarray and interfeature areas do not carry any chemical compound such as polynucleotide (or other biopolymer of a type of which the features are composed). Interarray areas and interfeature areas typically will be present where arrays are formed by the conventional in situ process or by deposition of previously obtained moieties, as described above, by depositing for each feature at least one droplet of reagent such as from a pulse jet (for example, an inkjet type head) but may not be present when, for example, photolithographic array fabrication processes are used. It will be appreciated though, that the interarray areas and interfeature areas, when present, could be of various sizes and configurations.

The devices and methods of the present invention are particularly useful in the preparation of individual substrates with oligonucleotide arrays for determinations of polynucleotides. As explained briefly above, in the field of bioscience, arrays of oligonucleotide probes, fabricated or deposited on a surface of a substrate, are used to identify DNA sequences in cell matter. The arrays generally involve a surface containing a mosaic of different oligonucleotides or sample nucleic acid sequences or polynucleotides that are individually localized to discrete, known areas of the surface. In one approach, multiple identical arrays across a complete front surface of a single substrate or support are used.

As mentioned above, biopolymer arrays can be fabricated by depositing previously obtained biopolymers (such as from synthesis or natural sources) onto a substrate, or by in situ synthesis methods.

The in situ method for fabricating a polynucleotide array typically follows, at each of the multiple different addresses at which features are to be formed, the same conventional iterative sequence used in forming polynucleotides from nucleoside reagents on a substrate by means of known chemistry. This iterative sequence is as follows: (a) coupling a selected nucleoside through a phosphite linkage to a functionalized substrate in the first iteration, or a nucleoside bound to the substrate (i.e. the nucleoside-modified substrate) in subsequent iterations; (b) optionally, but preferably, blocking unreacted hydroxyl groups on the substrate bound nucleoside; (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The functionalized substrate (in the first cycle) or deprotected coupled nucleoside (in subsequent cycles) provides a substrate bound moiety with a linking group for forming the phosphite linkage with a next nucleoside to be coupled in step (a). A number of reagents involved in the above synthetic steps such as, for example, phosphoramidite reagents, are sensitive to moisture and anhydrous conditions and solvents are employed. Final deprotection of nucleoside bases can be accomplished using alkaline conditions such as ammonium hydroxide, in a known manner.

The foregoing chemistry of the synthesis of polynucleotides is described in detail, for example, in Caruthers, Science 230: 281-285, 1985; Itakura, et al., Ann. Rev. Biochem. 53: 323-356; Hunkapillar, et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, and 5,869,643, EP 0294196, and elsewhere.

As mentioned above, various ways may be employed to produce an array of polynucleotides on the surface of a substrate such as a glass substrate. Such methods are known in the art. One in situ method employs inkjet printing technology to dispense the appropriate phosphoramidite reagents and other reagents onto individual sites on a surface of a substrate. Oligonucleotides are synthesized on a surface of a substrate in situ using phosphoramidite chemistry. Solutions containing nucleotide monomers and other reagents as necessary such as an activator, e.g., tetrazole, are applied to the surface of a substrate by means of thermal ink-jet technology. Individual droplets of reagents are applied to reactive areas on the surface using, for example, a thermal ink-jet type nozzle. The surface of the substrate may have an alkyl bromide trichlorosilane coating to which is attached polyethylene glycol to provide terminal hydroxyl groups. These hydroxyl groups provide for linking to a terminal primary amine group on a monomeric reagent. Excess of non-reacted chemical on the surface is washed away in a subsequent step. For example, see U.S. Pat. No. 5,700,637 and PCT WO 95/25116 and PCT application WO 89/10977.

Another approach for fabricating an array of biopolymers on a substrate using a biopolymer or biomonomer fluid and using a fluid dispensing head is described in U.S. Pat. No. 6,242,266 (Schleifer, et al.). The head has at least one jet that can dispense droplets onto a surface of a substrate. The jet includes a chamber with an orifice and an ejector, which, when activated, causes a droplet to be ejected from the orifice. Multiple droplets of the biopolymer or biomonomer fluid are dispensed from the head orifice so as to form an array of droplets on the surface of the substrate.

In another embodiment (U.S. Pat. No. 6,232,072) (Fisher) a method of, and apparatus for, fabricating a biopolymer array is disclosed. Droplets of fluid carrying the biopolymer or biomonomer are deposited onto a front side of a transparent substrate. Light is directed through the substrate from the front side, back through a substrate backside and a first set of deposited droplets on the first side to an image sensor.

An example of another method for chemical array fabrication is described in U.S. Pat. No. 6,180,351 (Cattell). The method includes receiving from a remote station information on a layout of the array and an associated first identifier. A local identifier is generated corresponding to the first identifier and associated array. The local identifier is shorter in length than the corresponding first identifier. The addressable array is fabricated on the substrate in accordance with the received layout information.

Substrates comprising polynucleotide arrays may be provided in a number of different formats. In one format, the array is provided as part of a package in which the array itself is disposed on a first side of a glass or other transparent substrate. This substrate is fixed (such as by adhesive) to a housing with the array facing the interior of a chamber formed between the substrate and housing. An inlet and outlet may be provided to introduce and remove sample and wash liquids to and from the chamber during use of the array. The entire package may then be inserted into a laser scanner, and the sample-exposed array may be read through a second side of the substrate.

In another format, the array is present on an unmounted glass or other transparent slide substrate. This array is then exposed to a sample optionally using a temporary housing to form a chamber with the array substrate. The substrate may then be placed in a laser scanner to read the exposed array.

In another format the substrate is mounted on a substrate holder and retained thereon in a mounted position without the array contacting the holder. The holder is then inserted into an array reader and the array read. In one aspect of the above approach, the moieties may be on at least a portion of a rear surface of a transparent substrate, which is opposite a first portion on the front surface. In this format the substrate, when in the mounted position, has the exposed array facing a backer member of the holder without the array contacting the holder. The backer member is preferably has a very low in intrinsic fluorescence or is located far enough from the array to render any such fluorescence insignificant. Optionally, the array may be read through the front side of the substrate. The reading, for example, may include directing a light beam through the substrate from the front side and onto the array on the rear side. A resulting signal is detected from the array, which has passed from the rear side through the substrate and out the substrate front side. The holder may further include front and rear clamp sets, which can be moved apart to receive the substrate between the sets. In this case, the substrate is retained in the mounted position by the clamp sets being urged (such as resiliently, for example by one or more springs) against portions of the front and rear surfaces, respectively. The clamp sets may, for example, be urged against the substrate front and rear surfaces of a mounted substrate at positions adjacent a periphery of that slide. Alternatively, the array may be read on the front side when the substrate is positioned in the holder with the array facing forward (that is, away from the holder).

Regardless of the specific format, the above substrates may be employed in various assays involving biopolymers. For example, following receipt by a user of an array made by an apparatus or method of the present invention, it will typically be exposed to a sample (for example, a fluorescent-labeled polynucleotide or protein containing sample) and the array is then read. Reading of the array may be accomplished by illuminating the array and reading the location and intensity of resulting fluorescence at each feature of the array. For example, a scanner may be used for this purpose where the scanner may be similar to, for example, the AGILENT MICROARRAY SCANNER available from Agilent Technologies Inc, Palo Alto, Calif. Other suitable apparatus and methods are described in U.S. patent applications: Ser. No. 09/846,125 “Reading Multi-Featured Arrays” by Dorsel, et al.; and Ser. No. 09/430,214 “Interrogating Multi-Featured Arrays” by Dorsel, et al. The relevant portions of these references are incorporated herein by reference. However, arrays may be read by methods or apparatus other than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature that is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

When one item is indicated as being “remote” from another, this is referenced that the two items are at least in different buildings, and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

In the above apparatus, the plurality of receptacles, the wet wash pad and the dry pad may be contained in a compartment in a housing. In the above apparatus, the mechanism may move the apparatus transversely to the droplet dispensing nozzles.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. An apparatus comprising:
(a) a substrate mount for receiving a substrate,
(b) a dispensing device for dispensing reagents for synthesizing a biopolymer on a surface of said substrate, and
(c) an optical system for positioning said substrate mount along said y-axis and an optical system for positioning said dispensing device along said x-axis, said optical systems cooperating to position said substrate mount and said dispensing device relative to one another,
wherein one of said substrate mount and said dispensing device is adapted for translation along a y-axis and for rotation about a central axis of the substrate mount that is parallel to a z-axis, and the other of said substrate mount and said dispensing device is adapted to move along an x-axis transversely to the direction of movement of said one.

2. An apparatus according to claim 1 further comprising a touch system for positioning said substrate and said dispensing device along a z-axis.

3. An apparatus according to claim 1 wherein said optical system for positioning said substrate mount comprises at least one image sensor and said substrate comprises at least one target image for imaging by said image sensor.

4. An apparatus according to claim 1 wherein said apparatus further comprises a calibration system, said optical systems and said calibration system cooperating to position said substrate mount along said y-axis and said dispensing device along said x-axis.

5. An apparatus according to claim 4 wherein said calibration system comprises a locator device having a predetermined fixed target location and a camera acting in cooperation with said optical systems.

6. An apparatus according to claim 2 wherein said substrate mount is adapted such that its orientation is adjusted to align said substrate along said y-axis as a result of input from said optical system for positioning said substrate mount and wherein said dispensing device is adapted such that its orientation is adjusted to align said dispensing device along said x-axis as a result of input from said optical system for positioning said dispensing device.

7. An apparatus according to claim 6 wherein said optical systems communicate with a computer, which provides input from said optical systems to said substrate mount and to said dispensing device and said touch system communicates with said computer.

8. An apparatus according to claim 1 further comprising a delivery device for delivering said substrate to said substrate mount, said delivery device having associated therewith a delivery device optical system for positioning said substrate to be within the field of view of the said support mount optical system.

9. An apparatus according to claim 8 wherein said delivery device optical system comprises at least one image sensor and said substrate comprises at least one target images for imaging by said image sensor.

10. An apparatus for synthesizing a plurality of biopolymer features on the surface of a substrate, said apparatus comprising:
(a) a substrate mount for receiving a substrate, said substrate mount being adapted for translation along an y-axis and for rotation about a central axis of the substrate mount that is parallel to a z-axis,
(b) a dispensing device for dispensing reagents for synthesizing biopolymers on a surface of said substrate wherein said dispensing device moves transversely with respect to said substrate mount,
(c) an optical system for positioning said substrate mount along said y-axis and an optical system for positioning said dispensing device along said x-axis, said optical systems cooperating to position said substrate mount and said dispensing device relative to one another,
(d) a touch system for positioning said substrate and said dispensing device along a z-axis,
(e) a loading station for loading said reagents into said dispensing device,
(f) a mechanism for moving said dispensing device and/or said loading station relative to one another,
(g) a wash station for washing said dispensing device, and
(h) a mechanism for moving said dispensing device and/or said wash station relative to one another.

11. An apparatus according to claim 10 further comprising an inspection device for inspecting the reagents dispensed to the surface of said substrate.

12. An apparatus according to claim 10 wherein said wash station comprises:
(a) a plurality of receptacles for sealingly engaging each head comprising said nozzles, said receptacles containing a wash solution,
(b) a wet wash pad for engaging a surface comprising said nozzles,
(c) a dry pad for engaging said surface, and
(d) a mechanism for moving said nozzles from said plurality of receptacles to said wet wash pad and then to said dry pad.

13. An apparatus according to claim 10 wherein said optical system for positioning said substrate mount comprises two or more image sensors and said substrate comprises a corresponding number of target images and said substrate comprises target images for imaging by said image sensors.

14. An apparatus according to claim 10 wherein said touch system comprises at least two opposing touch probes.

15. An apparatus according to claim 10 wherein said apparatus further comprises a calibration system, said optical systems and said calibration system cooperating to position said substrate mount along said y-axis and said dispensing device along said x-axis.

16. An apparatus according to claim 15 wherein said calibration system comprises a locator device having a predetermined fixed target location and a camera acting in cooperation with said optical systems.

17. An apparatus according to claim 10 wherein said substrate mount is adapted such that its orientation is adjusted to align said substrate along said y-axis as a result of input from said optical system for positioning said substrate mount and wherein said dispensing device is adapted such that its orientation is adjusted to align said dispensing device along said x-axis as a result of input from said optical system for positioning said dispensing device.

18. An apparatus according to claim 10 further comprising a delivery device for delivering said substrate to said substrate mount, said delivery device having associated therewith a delivery device optical system for positioning said substrate to be within the field of view of the said support mount optical system.

19. An apparatus according to claim 18 wherein said delivery device optical system comprises two or more image sensors and said substrate comprises a corresponding number of target images.

20. A method comprising:
(a) positioning a substrate along a y-axis by means of an optical system,
(b) positioning a dispensing device along an x-axis by means of an optical system, said optical systems cooperating to position said substrate mount and said dispensing device relative to one another,
(c) positioning said substrate and said dispensing device relative to one another along an orthogonal axis by means of at least one touch system, and
(d) depositing a reagent for synthesizing a biopolymer on a surface of said substrate by means of said dispensing device.

21. A method according to claim 20 wherein said optical system for positioning said substrate mount comprises two or more image sensors and said substrate comprises a corresponding number of target images.

22. A method according to claim 20 wherein said positioning of step (a) involves a calibration system, said optical systems and said calibration system cooperating to position said substrate mount along said y-axis and said dispensing device along said x-axis.

23. A method according to claim 22 wherein said calibration system comprises a locator device having a predetermined fixed target location and a camera acting in cooperation with said optical system.

24. A method according to claim 23 wherein adjustments are made to the orientation of said substrate along said x-axis as a result of input from said optical system and wherein adjustments are made to the orientation of said dispensing device and said substrate mount along said y-axis as a result of input from said optical system.

25. A method according to claim 20 wherein said system comprises touch probes that are aligned optically.

26. A method for synthesizing an array of biopolymers on a surface of a substrate, said method comprising, in multiple rounds of subunit additions, adding one or more polymer subunits at each of multiple feature locations on said surface to form one or more arrays on said surface, each round of subunit additions comprising:
(a) bringing said substrate and a dispensing system for dispensing said polymer subunits for the synthesis of said biopolymers into a dispensing position relative to said activated discrete sites on said surface wherein said substrate is positioned along a y-axis by means of an optical system and said dispensing system is positioned along an x-axis and said substrate mount and said dispensing system are positioned relative to one another by means of said optical systems and wherein said dispensing system is positioned along a z-axis by means of at least one touch system,
(b) dispensing said polymer subunits to said discrete sites, and
(c) removing said substrate and/or said dispensing system from said relative dispensing position.

27. A method according to claim 26 wherein said optical system for positioning said substrate mount comprises two or more image sensors and said substrate comprises a corresponding number of target images.

28. A method according to claim 26 wherein said positioning of step (a) involves a calibration system, said optical systems and said calibration system cooperating to position said substrate mount along said y-axis and said dispensing device along said x-axis.

29. A method according to claim 28 wherein said calibration system comprises a locator device having a predetermined fixed target location and a camera acting in cooperation with said optical system.

30. A method according to claim 29 wherein adjustments are made to the orientation of said substrate along said x-axis as a result of input from said optical system and wherein adjustments are made to the orientation of said dispensing device and said substrate mount along said y-axis as a result of input from said optical system.

31. A method according to claim 26 wherein said touch system comprises at least two opposing touch probes and said touch probes are aligned optically.

32. A method according to claim 26 wherein said biopolymers are polynucleotides or polypeptides.

33. A method according to claim 32 further comprising exposing the array to a sample and reading the array.

34. A method comprising forwarding data representing a result obtained from a reading of an array exposed according to the method of claim 33.

35. A method comprising transmitting to a remote location data representing a result of an interrogation obtained by reading of an array exposed according to the method of claim 33.

36. A method comprising receiving data representing a result of an interrogation obtained by reading of an array exposed according to the method of claim 33.

37. A method for washing droplet dispensing nozzles to remove residual reagents for synthesizing biopolymers, said method comprising:
(a) sealingly engaging each head comprising said nozzles having said residual reagents therein with a receptacle containing a wash solution,
(b) flushing each of said nozzles with said wash solution,
(c) disengaging each of said nozzles from respective receptacles,
(d) engaging a surface comprising said nozzles with a wet wash pad, and
(e) engaging said surface with a dry pad.

38. A method according to claim 37 further comprising repeating step (e) if residual wash solution is present.

39. A method according to claim 37 wherein step (d) is performed by wiping said surface over said wash pad.

40. A method according to claim 37 wherein step (e) is performed by wiping said surface over said dry pad.

41. An apparatus for washing droplet dispensing nozzles to remove residual reagents for synthesizing biopolymers, said apparatus comprising:
(a) a plurality of receptacles for sealingly engaging each head comprising said nozzles having said residual reagents therein, said receptacles containing a wash solution,
(b) a wet wash pad for engaging a surface comprising said nozzles, and
(c) a dry pad for engaging said surface, and
(d) a mechanism for moving said apparatus relative to said droplet dispensing nozzles such that said nozzles serially engage said plurality of receptacles, said wet wash pad and then said dry pad.

42. An apparatus according to claim 41 wherein said plurality of receptacles, said wet wash pad and said dry pad are contained in a compartment in a housing.

43. An apparatus according to claim 41 wherein said mechanism moves said apparatus transversely to said droplet dispensing nozzles.

44. An apparatus for loading reagents into a dispensing device, said apparatus comprising one or more reagent receptacles and a retractable cover disposed over said receptacles.

45. An apparatus for washing a reagent dispensing device, said apparatus comprising one or more wet wash pads for wet washing a surface or a portion of the reagent dispensing device and one or more dry pads for dry wiping the reagent dispensing device.

46. An apparatus comprising:
(a) a substrate mount for receiving a substrate,
(b) a dispensing device for dispensing reagents for synthesizing a biopolymer on a surface of said substrate, and
(c) a touch system for positioning said substrate and said dispensing device along a z-axis.

47. An apparatus according to claim 46 further comprising an optical system for positioning said substrate mount along said y-axis and an optical system for positioning said dispensing device along said x-axis, said optical systems cooperating to position said substrate mount and said dispensing device relative to one another, wherein one of said substrate mount and said dispensing device is adapted for translation along a y-axis and for rotation about a central axis of the substrate mount that is parallel to a z-axis, and the other of said substrate mount and said dispensing device is adapted to move along an x-axis transversely to the direction of movement of said one.
